SEQUENCE LISTING (5 sequences)

SEQ ID NO: 1 - DNA, 40 bp, Artificial sequence
  Primer
agacgtgtgctcttccgatctcggtgggaaagaggtagag

SEQ ID NO: 2 - DNA, 39 bp, Artificial sequence
  Primer
agacgtgtgctcttccgatctgtgtcccggacctccagt

SEQ ID NO: 3 - DNA, 40 bp, Artificial sequence
  Primer
agacgtgtgctcttccgatctctcgctagtccttagggga

SEQ ID NO: 4 - DNA, 39 bp, Artificial sequence
  Primer
agacgtgtgctcttccgatctcagggacacggacttcat

SEQ ID NO: 5 - DNA, 39 bp, Artificial sequence
  Primer
agacgtgtgctcttccgatctcatcccgactctcgtcac

(19) United States
(12) Patent Application Publication
(10) Pub. No.: US 2024/0011018 A1
(43) Pub. Date: Jan. 11, 2024

(54) MULTIPLEXED METHOD FOR ASSESSING GLOBAL OR GENOMIC LOCUS-SPECIFIC LEVELS OF CHROMATIN MODIFICATION

(71) Applicant: Epigenica AB, Stockholm (SE)

(72) Inventor: Simon Elsässer, Stockholm (SE)

(21) Appl. No.: 18/253,517

(22) PCT Filed: Dec. 2, 2021
(86) PCT No.: PCT/EP2021/084023

§ 371 Date: May 18, 2023

(30) Foreign Application Priority Data
Dec. 2, 2020 (EP) 20211177.9

(51) Int. Cl. C12N 15/10

(52) U.S. Cl. CPC C12N 15/1055; C12N 15/1065

(57) ABSTRACT

The invention provides methods for assessing the global levels of a plurality of different chromatin modifications in parallel in a plurality of samples. The methods disclosed herein also relate to assessing the levels of a plurality of different chromatin modifications, in a plurality of locations of interest within genome, in a plurality of samples. The methods are highly multiplexed, quantitative and involve chromatin immunoprecipitation and sequencing technology.

### TECHNICAL FIELD

The present invention relates to the field of assessing the level of chromatin modifications in genomes. In particular, the invention relates to assessing the levels of epigenetic modifications. Changes to said modifications may e.g. be caused by treatment of cells comprising said genomes with different compounds, and thus the methods of the invention can be used to access the effects of test compounds on levels of chromatin modifications. The invention relates to assessing both the global levels of chromatin modifications as well as the levels of chromatin modifications at specific loci, e.g. a specific gene promoter.

### BACKGROUND

A wide range of methodologies exist to profile epigenomic features in mammalian cells. Typically the methodologies to profile epigenomic features in mammalian cells fall into two categories. However, all these methodologies have several limitations.

One category of methods involve accurately quantifying DNA and histone PTM levels in bulk (globally). These include bottom-up or middle-down mass spectrometric methods and antibody-based methods (dot blots, western blot, immunofluorescence). Multiplexing has been achieved in antibody-based methodologies e.g. using the Luminex bead platform.

The second category of methods involve to acquire genome-wide profiles of an individual PTM or other features with up to base-pair resolution. Such methods include many variations of ChIP-Seq methods, in-situ profiling methods (CUT&RUN, CUT&Tag). Chromatin immunoprecipitation and sequencing (ChIP-seq) is a method that can be used to map histone modifications genome-wide. It can enable the identification of cell-type specific functional elements and epigenetic states. ChIP relies on fragmentation of native or fixed chromatin using enzymatic digestion or sonication followed by immunoprecipitation with a specific antibody. After purification of the DNA, a library for next-generation sequencing may be generated (ChIP-Seq).

In-situ profiling techniques on the other hand use a protocol akin of immunostaining in intact nuclei. A key limitation of all the techniques above is the fact that they are qualitative or semi-quantitative at best, unless a spike-in reference is added to every sample.

The need for quantitative workflows has been appreciated in recent years and available quantitative ChIP-Seq techniques. In contrast to histone modifications, quantitative methodologies are widely used to profile DNA methylation. Methylated cytosines can be detected and accurately quantified with base-pair resolution by chemical or enzymatic base conversion, namely by whole-genome bisulfite sequencing (WGBS) and EM-seq. Thus, it is not surprising that DNA methylation has been the predominant and almost exclusive readout to study epigenetic toxicity.

### High-Throughput Methods

While automated protocols for ChIP and WGBS exist (e.g. HT-ChIPmentation), they are always limited to one condition and one antibody per workflow. Even though workflows can be parallelized in multiwell-format or automatized in microfluidic chips, the number of workflows multiplies by condition and antibody, and the cost (reagents, plasticware and operation time) typically scales linearly to the number of workflows.

### Multiplexed Quantitative Methods

A number of multiplexed methods have been developed to increase throughput in epigenomic methods. For this, molecular barcodes are added to the chromatin fragments so that they can be pooled from several samples. Based on the barcode, next-gen sequencing reads can be demultiplexed during the analysis. A range of such barcoding-first method has been described. In principle, pooled workflows are intrinsically quantitative, although technical challenges have hampered the use of barcoding-first methods for quantitative studies. More recent barcoding methodologies include MINUTE-ChIP, relying on unique molecular identifiers for accurate quantification (Kumar et al., 2019). Notably, MINUTE-ChIP is the only technology to date that has been shown to produce accurate quantitative comparisons over a large dynamic range. As described by Kumar et al., 2019, MINUTE-ChIP relies on sequencing a very large number of fragments. Thus, the number of sequenced fragments per sample and antibody in Kumar et al., 2019 is in the range of 6,065,348 to 32,979,288. Furthermore, MINUTE-ChIP as described by Kumar et al., 2019 is restricted to parallel analysis of only a low number of samples.

Probing the epigenome in response to drugs: It is well known that in a multicellular organisms, chromatin modifications may be characteristic for cell identity, differentiation state, and/or metabolic state of the cell, i.e. cell with similar cell identify, differentiation state and metabolic state often share similar patterns of chromatin modifications, whereas unrelated cells exhibit divergent chromatin modifications. Drugs interact with cells in our human body in diverse ways. By design or serendipity, many drugs have effects on the epigenome, impacting epigenetic gene regulatory mechanisms, cell fitness and/or cell identity. While it remains to be determined in each individual case if alterations would have a functional, potentially long-term, effect, it is relatively likely that any drug affects some global or local levels of histone PTMs or DNA modifications. This is because many common drug (side)-effects ultimately impinge on pathways that regulate the epigenome: as on or off-target, drugs may target signaling pathways that impinge on transcription factors, proteins that, by sequence-specific binding, orchestrate epigenomic landscapes. They may alter metabolism, e.g. by causing oxidative stress. Through providing substrates for epigenomic PTMs such as acetylation, phosphorylation or methylation, metabolic pathways directly influence global levels of many PTMs through mass action. DNA or histone modifying enzymes are target of many drugs, again directly influencing the epigenomic landscape. Finally, epigenetic features,e.g. the DNA methylation pattern on a tumor suppressor gene promoter, are thought to be intrinsically more heterogeneous than the genetics. Cytotoxic agents put selective pressure on cells, which may lead to a stabilization and expansion of epigenetic states connected to a survival phenotype that preexist as natural variation. The resulting epigenomic clonal population may persist to be more resistant to subsequent drug treatments.

The issue of performing epigenomic efficacy and toxicology studies lies in the fact that there is no unifying, simple readouts for epigenomic alterations. Epigenomic profiling has not been established as a part of high-throughput drug characterization and there is a clear need for methods in this specific sector. Detecting epigenetic side effects in cell-based assays could be used to filter out drug candidates early enough in the process to reduce animal experiments and clinical trials on compounds that show epigenetic toxicity.

### SUMMARY

The present disclosure provides methods that can be applied to measure both immediate and long term epigenetic alterations after drug treatment.

Thus, the present disclosure relates to methods for assessing the global levels of a plurality of different chromatin modifications in parallel in a plurality of samples. The methods disclosed herein also relate to assessing the levels of a plurality of different chromatin modifications, in a plurality of locations of interest within genome, in a plurality of samples. The methods are highly multiplexed, quantitative and involve chromatin immunoprecipitation and sequencing, preferably massive parallel sequencing.

Surprisingly, the invention shows that global levels of chromatin modification can be accurately determined even if highly multiplexed libraries are sequenced at a very low depth. Thus, it is sufficient to sequence in the range of 100 to 100,000 gDNA fragments per initial sample for each chromatin modification in order to be able to retrieve quantitative information on the global levels of a particular chromatin modification. Despite the resulting genomic coverage being extremely scarce, the invention shows an accurate correlation between the level of chromatin modifications of a sub-sample and the global levels under every condition tested. Accordingly, the methods of the invention are in general methods for quantitatively assessing the levels of a plurality of chromatin modifications. Thus, the invention shows that analysing only a subset of a genome allows accurate quantification of the global level of any chromatin modification under any given condition.

Thus, it is one aspect of the invention to provide methods of assessing the levels of a plurality of chromatin modifications in parallel in a plurality of samples, said method comprising the steps of
a. providing a plurality of test samples comprising chromatin from a cell population comprising a plurality of cells, wherein said samples are physically separated from each other,
b. fragmenting chromatin of each sample into chromatin fragments, wherein each chromatin fragment comprises a double-stranded genomic DNA (gDNA) fragment and optionally associated proteins,
c. tagging at least a fraction of the gDNA fragments within each sample with an ID-tag, wherein said ID-tag is an oligonucleotide which comprises a barcode sequence and optionally a unique molecular identifier (UMI) sequence, wherein each ID-tag comprises a different UMI sequence and optionally additional sequences, wherein gDNA fragments within one sample is tagged with a ID-tag comprising the same barcode sequence, and wherein different barcode sequences are used for each sample,
d. combining said tagged chromatin fragments generating a pool of tagged chromatin fragments,
e. providing a plurality of different antibodies, each specifically binding a chromatin modification,
f. incubating each antibody with said pool of tagged chromatin fragments or a random sub-pool thereof,
g. obtaining chromatin fragments binding each antibody, thereby obtaining a sub-pool comprising tagged gDNA fragments from chromatin fragments comprising the chromatin modification recognised by said antibody referred to as a “chromatin modification sub-pool”,
h. optionally amplifying at least a fraction of said tagged gDNA fragments in said chromatin modification sub-pool, thereby obtaining copies of gDNA fragments, wherein said gDNA fragments and said copies thereof collectively are referred to as “gDNA fragments”;
i. Randomly selecting in the range of n times 100 to 100,000 tagged gDNA fragments, from each chromatin modification sub-pool, wherein n is the number of samples provided in step a.; or
   pooling tagged gDNA fragments from all chromatin modification sub-pools into a combined pool and randomly selecting in the range of n times m times 100 to 100,000 tagged gDNA fragments from said combined pool, wherein n is the number of samples provided in step a. and m is the number of chromatin modification sub-pools;
j. Sequencing at least part of each of said selected tagged gDNA fragments, and determining the number of unique tagged gDNA fragments comprising each barcode sequence from each chromatin modification sub-pool, wherein
   i. at least the first barcode sequence and
   ii. the UMI sequence and/or a part of the gDNA sequence is sequenced, and wherein a unique tagged gDNA fragments comprises either a unique UMI and/or a unique gDNA sequence,
k. calculating the frequency of gDNA fragment comprising each barcode sequence within each chromatin modification sub-pool,
   wherein a higher frequency of gDNA fragments comprising a barcode sequence indicates a higher level of said chromatin modification in the sample tagged with ID-tags comprising said barcode sequence.

The invention also provides methods of determining the influence of test compounds on the level of a plurality of chromatin modifications, said method comprising the steps of
a. Providing one or more test compounds;
b. Cultivating a plurality of cells in the presence of said test compounds or combinations thereof, wherein cells cultivated in the presence of different test compounds or combinations therof are physically separated from each other, and wherein cells cultivated in the presence of a given test compound or combination thereof is a cell population;
c. Performing the methods of assessing the levels of a plurality of chromatin modifications according to the invention, wherein each test samples comprises chromatin from different cell populations.

The influence of test compounds on the level of a plurality of chromatin modifications levels of various chromatin modifications may be determined by comparing the levels of said chromatin modifications in a cell population after cultivation in the presence of test compound(s) with a control, e.g. a cell population cultivated in the absence of the test compound(s).

### DESCRIPTION OF DRAWINGS

FIG. 1 shows a schematic overview of an example of the methods disclosed herein. In this example samples 1, 2, 3, 4 . . . 99 are each subjected to lysis followed by fragmentation of chromatin and addition of an ID-tag comprising a first barcode sequence unique for each sampple. The tagged chromatin fragments are pooled and divided into sub-pools. One sub-pool is the input sub-pool, whereas the other sub-pools are subjected to chromatin immunoprecipitation (ChIP) with various antibodies named ChIP1, ChIP2, ChIP3 . . . ChIP15. The gDNA fragments obtained after ChIP are tagged with a second tag comprising a second barcode sequence unique for each ChIP, amplified and otherwise made ready for massive parallel sequencing according to the specific requirement of the equipment used for sequencing. The fragments are pooled and only a random fraction of the fragments is sequenced. The number of unique sequences (also termed “unique reads”) tagged with each combination of first barcode and second barcode is determined, and for each second barcode the percentage of unique reads tagged with each first barcode is determined as illustrated by the pie diagrams, wherein one pie diagram represents the frequency of unique counts for each sample in a given ChIP. Similarly, the frequency of unique reads tagged with each first barcode sequence is determined for the input. The frequency of each first barcode after each ChIP is normalised to the frequency in the Input.

FIG. 2 Schematic comparing global and locus-specific quantitation with different methods. 1) Method generating detailed quantitative genomic landscapes using deep sequencing of tens of millions to hundreds of millions of chromatin fragments (referred to as MINUTE-ChIP). 2) a method according to the invention where only 1000 to 10,000 randomly selected chromatin fragments are sequenced generates the same accurate quantitation (referred to as hmqChIP) 3) a method according to the invention using locus-specific primers, a small fraction of the chromatin fragments is selected and the selection of fragments yields a quantitation of the local levels at one or many loci instead of a quantitation of the global average.

FIG. 3 shows in principle an example method according to the invention. Chromatin fragments from sample of cells cultivated under Condition A and B are tagged using the ID-tag A and B, respectively. Typically, only a fraction of chromatin fragments in from each sample are tagged. Typically, the number of tagged chromatin fragments is not exactly the same in Condition A and B. Chromatin fragments from conditions A and B are pooled and an aliquot of the pool (a sub-pool of randomly selected fragments) is subjected to chromatin immunoprecipitation (ChIP) using an antibody against a specific chromatin modification, here Histone H3 Lysine 27 trimethylation (H3K27me3). Any chromatin fragments carrying the H3K27me3 modification compete for antibody binding sites with the same affinity, irrespective of the presence of a tag or the sequence of the tag, i.e. origin of the chromatin fragment. After washing away unbound molecules, the molecules retained by the antibody in principle all have a H3K27me3 modification. Crucially, because of the equal probability for any H3K27me3-modified molecule to be captured by the antibody, the selection of chromatin fragments in the chromatin modification sub-pool represents the product of the probabilities of chromatin fragments being tagged and having the H3K27me3 modification, irrespective of which condition they originate from. A small random subset of molecules from input pool and the chromatin modification sub-pool are sequenced, and the number of A and B barcodes are determined in the input and chromatin modification sub-pool. In the present example, A and B barcodes are sequenced at 1:1 ratio in the input pool. A and B barcodes are sequenced at 2:1 ratio in the chromatin modification sub-pool. As a result, it can be deduced that Condition A has exactly twice as much H3K27me3 modified chromatin fragments as Condition B. Or in other words, the global level of H3K27me3 in Condition B is thus 50% of that in condition A.

FIG. 4 shows an example of calculation of the relative quantity of a given chromatin modification after a given treatment compared to a control condition. Three replicates (R1, R2, R3) samples are prepared for a specific treatment and control (e.g. cells cultivated in the presence or absence of a test compound). Chromatin fragments are tagged with six different ID-tags, comprising six different first barcodes. A The barcode representation in the input sub-pool is determined by sequencing a small random subset of the input pool and counting unique fragments B The barcode representation of each chromatin modification sub-pool is determined by sequencing a small random subset of the fragments of each chromatin modification sub-pool and counting unique fragments. C The count of each barcode in each chromatin modification sub-pool is divided by the corresponding count of each barcode in the input sub-pool, yielding the input-normalized unique read count (INRC) for each sample. The INRC reflects the abundance of each first barcode in the chromatin modification sub-pool, corrected by the abundance of each first barcode in the input sub-pool. Because the probability of binding of modified nucleosomes to the available antibody binding sites is independent of the ID-tag as exemplified in FIG. 3, the INRC is linearly related to the global enrichment of the probed chromatin modification in the respective sample. The global level of the probed chromatin modification in the treatment samples as compared to the control can be deduced by dividing the treatment INRC by the control INRC.

FIG. 5 shows that methods where only a small fraction of fragments are sequenced (referred to as hmqChIP) is as quantitative as methods where a large amount of fragments are sequenced (referred to as MINUTE-ChIP) even though hmqChIP may involve ˜10,000 times less sequencing reads. hmqChIP allows accurate quantification of relative histone H3K27me3 modification levels by sampling a very small number of unique reads. Data was generated as described by Kumar and Elsässer, 2019, except that only a small number of randomly selected sequences were used. A standard curve was generated by preparing samples with pre-set quantities of histone H3K27me3, mixing a cell source with maximal levels of H3K27me3 with a cell source depleted of H3K27me3 at known ratios (7 different ratios in duplicates, corresponding to 14 samples). 2,500, 25,000 and 250,000 sequences were randomly selected from the chromatin modification sub-pool and input sub-pool each corresponding to—in theory—approx. 179, 1786 and 17,857 sequences per sample. Unique chromatin fragments containing each of the 14 barcodes were counted in the chromatin modification and input sub-pools. The input-normalized unique read counts were calculated by determining the ratio of chromatin modification reads versus input reads for each first barcode. A standard curve was calculated by linear regression analysis, using said 250,000, 25,000 or 2,500 reads from the chromatin modification sub-pool. The measurement is linearly correlated with, and proportional to, the pre-set quantity. The R² value, an indicator of how closely the quantities data predicts the true quantities, remains extremely good (>0.98) even when using only 2,500 reads in total to produce the standard curve.

FIG. 6 shows the accurate quantification of relative histone H3K27me3 modification levels by methods where only a small fraction of fragments are sequenced (refererd to as hmqChIP). Data was generated as described by Kumar and Elsässer, 2019, except that only a small number of randomly selected sequences were used. The dataset contains a comparison of mouse embryonic stem cells in a untreated control condition (“untreated”) and a condition where the cells have been treated with two inhibitors (“2i”), each present in biological triplicates. Two analyses were performed: In the top analysis, mapping to the corresponding reference genome was used to determine uniqueness of the chromatin fragments analyzed. In the bottom analysis, no mapping to a reference genome was performed and instead the UMI sequence was used to determine the count of unique chromatin fragments. For each analyses, a defined number of sequences were randomly extracted from the dataset as indicated on the X axis. On the Y axis, the quantity of H3K27me3 relative to the “untreated” control is given. Error bars show standard deviation of the triplicate measurements. Significance was calculated using a two-sided t-Test and indicated as follows: n.s. is non-significant; * is p<0.05; ** is p<0.01. Kumar and Elsässer reported a 2.3fold increase in H3K27me3 upon “2i” treatment based on sequencing more than tens of millions of chromatin fragments. The analyses show that accurate (2.3-fold difference) and confident (statistically significant) quantification is possible with as little as in the range of 1000 to 10,000 mapped reads or UMI counts in total for the six replicates. The limit of hmqChIP shown in this example lies at approximately ˜200 unique reads per condition.

FIG. 7 shows an example of how gDNA fragments may be tagged, amplified by PCR and sequenced in methods of the invention for determining global levels of chromatin modifications 1) ID-Tag is added randomly to the gDNA moiety of a fraction of all chromatin fragments from each sample. The ID-tag comprises an amplification sequence, a UMI and first barcode sequence (BC). The ID-tag comprises one non-ligatable end 2) After pooling, splitting pool into sub-pools, and chromatin immunoprecipitation, gDNA fragments of each chromatin modification sub-pool are purified. 3) a second tag is ligated onto the gDNA fragment. The second tag comprises an amplification sequence and may optionally comprise a second barcode sequence. The non-ligatable terminus of the ID-tag ensures that second tag is not ligated onto the ID-tag. 4) The double tagged gDNA fragments are amplified by PCR using primers specific for amplification sequence of the ID-tag and amplification sequence of second tag. Sequencing platform-specific adaptors may be added with the primer sequences. 5) The UMI-BC-gDNA part is sequenced.

FIG. 8 shows an example of how gDNA fragments may be tagged, amplified by PCR and sequenced in methods of the invention for determining local levels of chromatin modifications 1) ID-Tag is added randomly to the gDNA moiety of a fraction of all chromatin fragments from each sample. The ID-tag comprises an amplification sequence, a UMI and first barcode sequence (BC). 2) After pooling, splitting pool into sub-pools, and chromatin immunoprecipitation, gDNA fragments of each chromatin modification sub-pool are purified. 3) The ID-tagged gDNA fragments are amplified by PCR using one primer specific for amplification sequence of the ID-tag and a primer specific one or multiple loci of interest. Sequencing platform-specific adaptors may be added as part of the primers. Primers may also optionally comprise a second barcode sequence. 4) The UMI-BC-gDNA part is sequenced.

FIG. 9 shows an example of how gDNA fragments may be tagged, amplified by linear amplification and PCR and sequenced in methods of the invention for determining global levels of chromatin modifications 1) ID-Tag is added randomly to the gDNA moiety of a fraction of all chromatin fragments from each sample. The ID-tag comprises an amplification sequence, an RNA polymerase promoter, a UMI and first barcode sequence (BC). The ID-tag comprises one non-ligatable end 2) After pooling, splitting pool into sub-pools, and chromatin immunoprecipitation, gDNA fragments of each chromatin modification sub-pool are purified. 3) ID-tagged gDNA are transcribed and amplified using the RNA Polymerase promoter embedded in the ID-tag. 4) A second tag is ligated onto the RNA copies of the gDNA fragment. The second tag comprises an amplification sequence and may optionally comprise a second barcode sequence. 5) The RNA is reverse transcribed using a primer complementary to the second tag. 6)

The double tagged DNA fragments are amplified by PCR using primers specific for amplification sequence of the ID-tag and amplification sequence of second tag. Sequencing platform-specific adaptors may be added with reverse transcription or PCR primer sequences. 7) UMI-BC-gDNA part is sequenced.

FIG. 10 shows an example of how gDNA fragments may be tagged, amplified by linear amplification and PCR and sequenced in methods of the invention for determining local levels of chromatin modifications. 1) ID-Tag is added randomly to gDNA part of a fraction of all chromatin fragments from each sample. The ID-tag comprises an amplification sequence, an RNA polymerase promoter, a UMI and first barcode sequence (BC). 2) After pooling, splitting pool into sub-pools, and chromatin immunoprecipitation, gDNA fragments of each chromatin modification sub-pool are purified. 3) ID-tagged gDNA are transcribed and amplified using the RNA Polymerase promoter embedded in the ID-tag. 4) The RNA is reverse transcribed using a primer carrying complementary sequences to one or multiple loci of interest. A second tag may be added as part of the primer sequence. 5) The double tagged DNA fragments are amplified by PCR using primers specific for amplification sequence of the ID-tag and amplification sequence of second tag. Sequencing platform-specific adaptors may be added with reverse transcription or PCR primer sequences. 6) UMI-BC-gDNA part is sequenced.

FIG. 11 shows an hmqChIP experiment for quantifying drug effects on three histone modifications: (A) shows the scheme of the experiment. INRCs using DMSO controls as reference were calculated according to the method and plotted in a heatmap with grayscale and circle size reflecting the relative quantity of the modifications. (B) Results of first sequencing. (C) results of second sequencing run using 25 Mio reads. (D) results of second sequencing run using 1 Mio randomly selected reads out of 25 Mio.

FIG. 12 shows the results of an hmqChIP experiment for quantifying drug effects on two histone modifications on a single locus, using locus-specific primers hybridizing with the CDKN1A (p21) locus for library preparation. Libraries from two antibodies against two different histone modifications, H3K27ac and H3K9me3 and Input were used to quantify the effect of four different samples treated as indicated. INRCs using DMSO controls as reference were calculated according to the method and plotted in a barplot with the replicate datapoint shown as dots.

### DETAILED DESCRIPTION

### Definitions

In this specification, unless otherwise specified, “a” or “an” means “on or more”.

As used herein the term “approximately” when used in relation to a numerical value refers to +/−5%, more preferably +/−1%.

As used herein the term “epigenetic modification” refers to modifications within chromatin that are stable and heritable through cell division.

As used herein the term “global levels” refers to the average density or average relative levels of a given chromatin modification across the whole genome.

As used herein the term “local levels” refers to the average density or average relative levels of a given chromatin modification across one specified locus of interest, such as within a 1000 bp interval within the genome, or within a plurality of loci of interest.

The term “chromatin” as used herein refers to a complex of genomic DNA (gDNA) and protein found in the nucleus of cells. The primary protein component of chromatin is made up of histones.

### Method

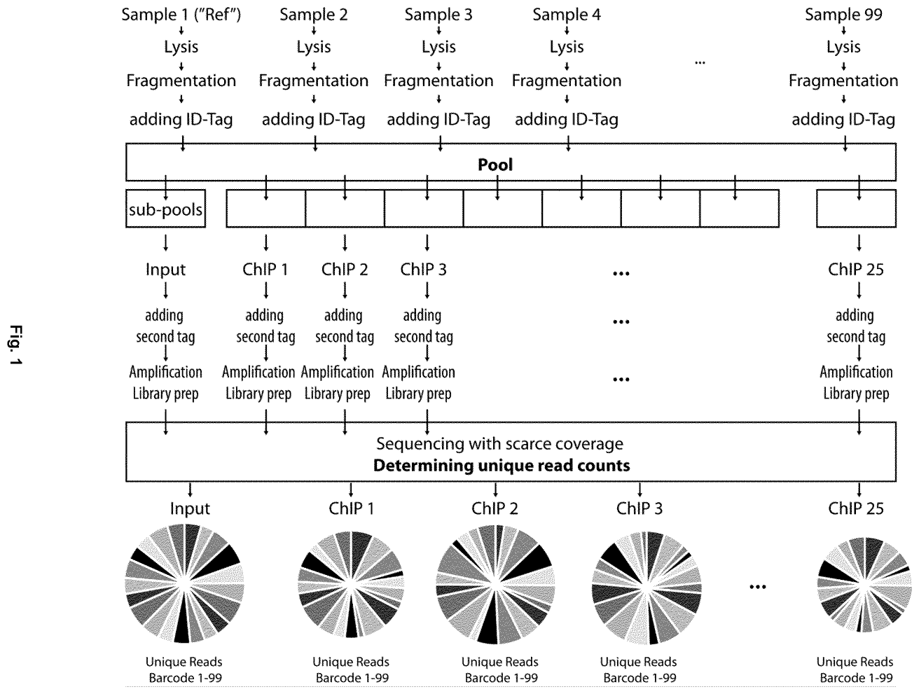

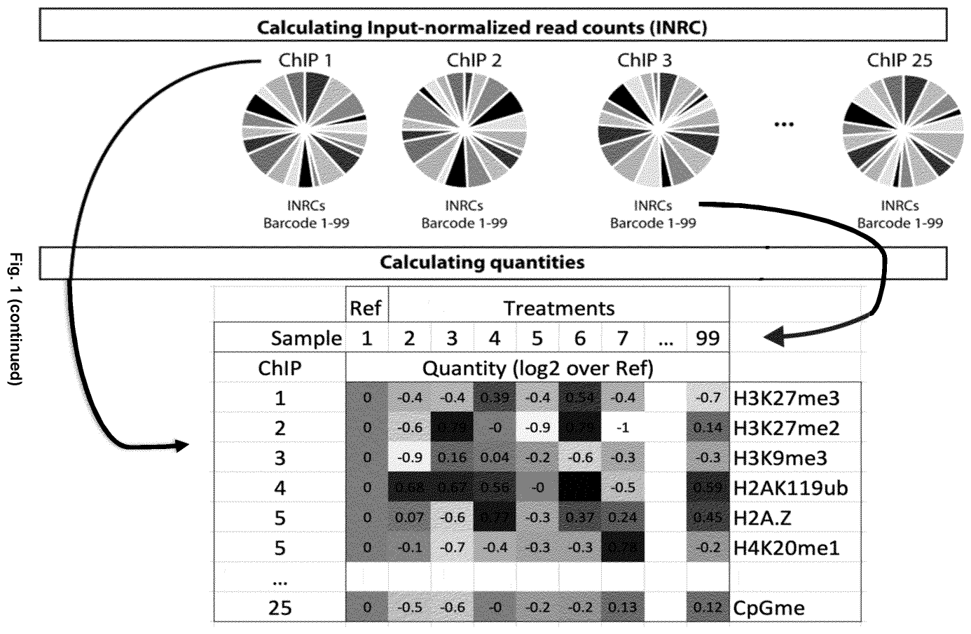

The disclosure describes methods of assessing levels of chromatin modifications in parallel in a plurality of samples. The methods are useful for assessing either the global levels or local levels of a plurality of chromatin modifications in a plurality of samples in parallel. In particular, the methods are useful for determining said levels relative to each other or relative to a control. The present disclosure relates to a highly multiplexed, quantitative, technology to measure the relative global or local levels of a plurality of chromatin modifications, such as histone posttranslational modifications (PTMs) or nucleotide modifications, in a multitude of different samples. An example of a useful workflow of the method is schematically described in FIG. 1.

In particular, the methods of the invention are useful for determining in a quantitative manner, the gain or reduction of a given chromatin modification compared to a control. The method allows parallel analysis of multiple samples, and thus the method can be used for quantitative analysis of the enrichment or reduction of a plurality of given chromatin modifications in different samples.

In order to allow quantitative determination of levels of chromatin modifications across different samples, it is important that said samples are treated in the same manner. Thus, it is preferred that all samples are treated in essentially the same manner. It is also preferred that all chromatin modification sub-pools are treated in the same manner.

In preferred embodiments, the methods can be used for determining the effect of one or more test compounds on the global and/or local level of one or more chromatin modification. In such embodiments, cells are incubated with different test compounds or combinations thereof, and the global and/or local level of a plurality of chromatin modifications in the cells is determined by the methods of the invention. In that manner the effect of the various test compounds on the epigenome can be assessed.

### Chromatin Modification

The present disclosure describes methods for assessing global levels of a plurality of chromatin modifications.

As used herein the term “chromatin modification” refers to any particular feature of chromatin, which is desirable to determine the level of. Frequently, the chromatin modification may be an epigenetic modification.

The chromatin modifications may for example be selected from the group consisting of
i. a protein bound to gDNA within the chromatin fragment
ii. a post-translational modification
iii. a modification of a nucleobase
iv. presence of a non-natural nucleo-base in the gDNA fragment
v. presence of a protein fragment produced through post-translational processing
vi. a non-canonical DNA structure

The methods allow simultaneous assessment of a plurality of different chromatin modifications, which may be any chromatin modification. Thus, the methods may involve assessment of a plurality of similar chromatin modifications or very different chromatin modifications. Thus, the methods may involve assessing a mixture of the different kinds of chromatin modifications described above.

The methods involve use of binding molecules, preferably antibodies specifically binding a particular chromatin modification. Thus, the chromatin modification may be any modification, which can be specifically recognised by binding molecule, preferably an antibody.

One or more of the chromatin modifications, which may be assessed by the methods of the invention may be epigenetic modifications. The term “epigenetic modification” as used herein refers to stable and heritable modifications. Said modifications are often chemical marking of chromatin. Epigenetic marks can include modification of gDNA as well as various post-translational modifications of proteins associated with gDNA, such as histones, e.g. any of the modifications described below in the section “Posttranslational modifications”.

Parent-of-origin-specific gene expression (either from the maternal or paternal chromosome) is often observed in mammals and is typically due to epigenetic modifications. In the parental germlines, epigenetic modification can lead to stable gene silencing or activation. Other epigenetic modifications may include a change in epigenetic state, chromatin structure, transcription, mRNA splicing, post-transcriptional modification, mRNA stability and/or half-life, translation, post-translational modification, protein stability and/or half-life and/or protein activity of at least one component of a cellular pathway associated with cancer.

As noted above, the chromatin modification may for example be a modification of a nucleotide, in particular a modification of a nucleotide within gDNA. Said modification may for example be methylation, e.g. methylation of a nucleobase. Modified nucleotides may for example be 5-methyl-cytosine, 5-hydroxymethyl-cytosine, 5-formylated cytosine, 5-carboxycytosine and 6-methyl-adenine.

As noted above, the chromatin modification may for example be the presence of one or more non-natural nucleobases. Non-limiting examples of non-natural nucleobases incldue 5-brom-2′-deoxiuridin and 5-Ethynyl-2′-deoxyuridine base.

As noted above, the chromatin modification may for example be presence of one or more non-canonical DNA structures. Non-limiting examples of non-canonical DNA structures include a G4 structure, a single-stranded DNA, and a RNA:DNA hybrid.

The methods of the invention may be for determining the global and/or local levels of at least one, preferably at least 2, such as at least 3, for example at least 5, such as at least 10, for example at least 15,such as in the range of 5 to 100, for example in the range of 5 to 50, such as in the range of 10 to 100, for example in the range of 10 to 50 chromatin modifications. In particular, it is preferred that at least one, preferably at least 2, such as at least 3, for example at least 5, such as at least 10, for example at least 15 of said chromatin modifications are posttranslational modifications of protein(s) (PTMs), e.g. any of the PTMs described in the section “Posttranslational modifications (PTMs)” below.

### Posttranslational Modifications (PTMs)

As used herein, the term “post-translational modification” (PTM) means a modification in the structure of a protein after its translation. The PTM may comprise addition of a chemical group, including but not limited to carboxylation, methylation, hydroxymethylation, acetylation, glutamylation, citrullination, phosphorylation, or glycosylation. The PTM may comprise an isomerization, including but not limited to proline isomerization, or formation of atypical isoaspartyl.

Examples of PTMs include, but are not limited to lysine mono-, di-, tri- methylation; acetylation, propionylation, butyrylation, crotonylation, isobutyrylatinon, ubiquitination, sumoylation, neddylation, glutarylation of lysine; serine phosphorylation, threonine phosphorylation; histidine phosphorylation; citrulline; arginine monomethylation or symmetric or asymmetric dimethylation.

In relation to chromatin modifications, the PTM is frequently a posttranslational modification of a histone (also referred to as “histone modification” herein). Said PTM of histones may be any of the aforementioned PTMs. For example, the histone modification, may be acetylation, methylation, demethylation, phosphorylation, adenylation, ubiquitination, or ADP ribosylation of one or more histones.

Said histone may for example be a histone selected from the group consisting of
a. Histone H3
b. Histone H3.1, H3.2, H3.3
c. Histone H3.X, H3.Y
d. Histone H4
e. Histone H2A
f. Histone H2A.X
g. Histone H2A.Z
h. Histone H2A.Z.1
i. Histone H2A.Z.2
j. Histone macroH2A
k. Histone H2A.Bbd; and
l. Histone H2B

Non-limiting example of histones with post translational modification include, but are not limited to
a. H3K4me1;
b. H3K4me2;
c. H3K4me3;
d. H3K79me3;
e. H3K9me1;
f. H3K9me2;
g. H3K9me3;
h. H3K27me1;
i. H3K27me2;
j. H3K27me3;
k. H4K20me1;
l. H4K20me2 and
m. H4K20me3.

The wealth of known histone posttranslational modifications (PTMs) and combinations thereof highlight the verbose coding potential of chromatin for epigenetic information.

It is well established that the disordered histone tails serve as signaling platforms for enzymes to write or erase marks, as well as readers/effectors that recognize a specific mark or combination of marks and link its appearance at a genomic locus to a functional outcome such as gene activity. The complexity and density of PTMs on histones provides a challenge for dissecting the impact on perturbations introduced to a cell, be it by endogenous signaling, exogenous stress, environmental factors or drugs.

### Global and Local Levels

The term ‘global level’ as used herein refers to the total average levels or average density of a specific chromatin modification across an entire genome. Said levels are most frequently determined as relative levels in comparison to a control. By way of example, if the chromatin modification is H3K4me3, the ‘global level’ is equivalent to the average level or density of said modification in the genome.

Global levels determined by the methods described herein are, in principle, equivalent to global levels determined by other biochemical methods, such as quantitative western blot or quantitative immunofluorescence microscopy. Global levels are typically provided as relative levels compared between a plurality of samples, determining the fold-change in global levels of one sample against another sample, which preferably may be a reference sample, more preferably a reference sample with a known quantity of the chromatin modification. The reference sample may for example be a synthetic sample, such as a preparation of gDNA fragments carrying histone protein with 100% density of a given chromatin modification. By using the quantitative comparison provided by the present invention, a previously unknown quantity of a given chromatin modification in a test sample can be calculated by considering the fold-change difference observed for the test sample relative to the reference sample. If the reference sample has a known quantity, the exact level may be calculated. Such calculation is accurate if the measurement produced by the quantitative method is linearly related or proportional to the true quantities in the sample. As described in detail herein, the methods of the present invention are indeed linearly related or proportional to the true quantities in the sample.

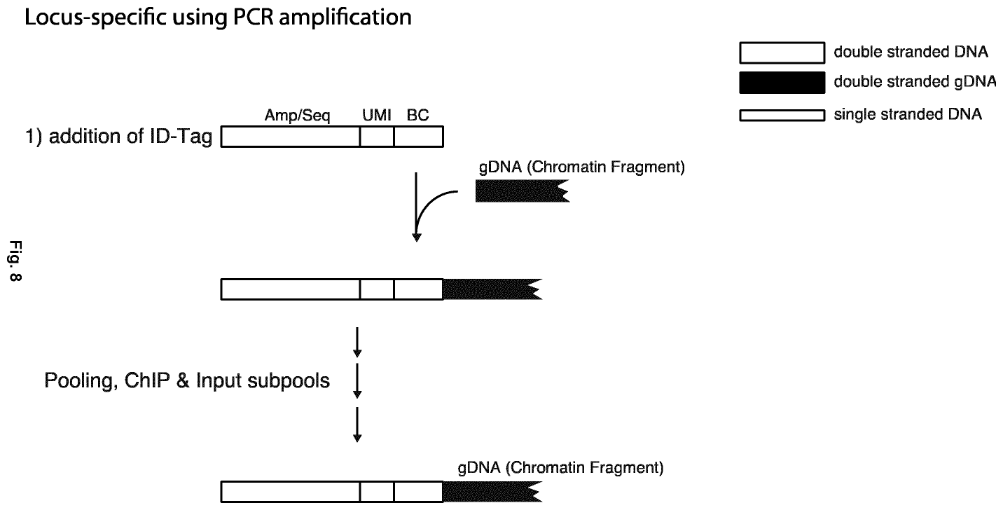

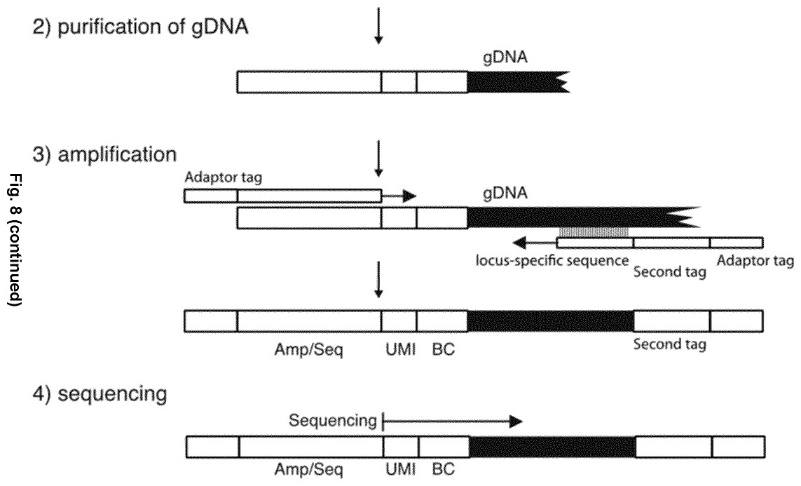

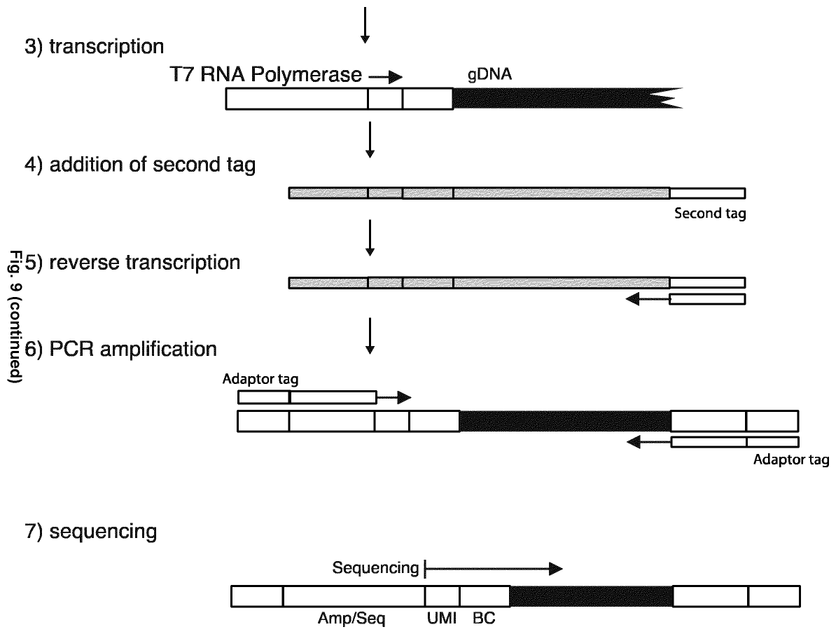

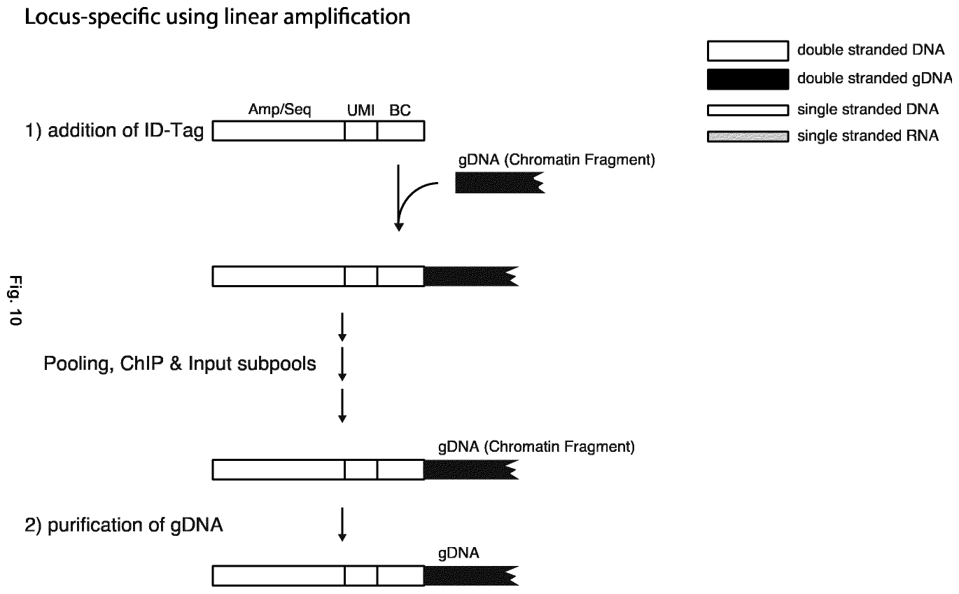

The methods disclosed herein comprises a step of randomly selecting in the range of 100 to 100,000 tagged gDNA fragments per sample for sequencing. In embodiments of the invention, wherein global levels are determined, said gDNA fragments may in principle to randomly selected from all gDNA fragments within the chromatin modification sub-pools and input sub-pool. Frequently, the methods disclosed herein comprise a step of amplification of gDNA fragments from the chromatin modification sub-pools and input sub-pool. In embodiment of the invention, where the methods are for determining the global levels of a plurality of chromatin modifications, said step of amplification is designed such that all gDNA fragments in principle have the same probability of amplification. This may for example be achieved by tagging a random fraction of the gDNA fragments an ID-tag comprising an amplification sequence and with a second tag comprising another amplification sequence in any of the manners described below. The amplification may then be made with primers recognising said amplification sequence, and will in principle be independent on the sequence of the gDNA fragment. Useful methods for tagging and amplification of gDNA fragments for assessing global levels of chromatin modification(s) are provided in FIGS. 7 and 9. The skilled person will be able to adapt the principles disclosed in those figures to other useful tagging and amplification methods.

In one embodiment, the global levels in a sample are not measured directly but approximated by assessing a plurality of local levels, preferably of a large number of loci, such as more than 100, or more than 1000 loci, that together provide representative subsample of the entire genome.

The term ‘local levels’, as used herein refers to the level (or density per a specified genomic length, e.g. in kilobases) of a given chromatin modification in a specific genomic location, such as within a 1000 base pair interval. Thus, typically the local level is the level of a given chromatin modification in a given locus. By way of example, the local level could refer to the level of H3K4me3 in the MYC gene promoter, as defined by an interval spanning 1000 base pairs around the MYC gene transcription start site.

Whereas the “local level” may be determined in one specific locus, it is comprised within the invention that local levels at more than one locus is determined simultaneously. Thus, the methods may be methods for determining the local level at least 2 loci, such as at least 5 loci, for example at least 10 loci, such as in the range of 2 to 100 loci, for example in the range of 2 to 50 loci, such as in the range of 2 to 25 loci, for example in the range of 5 to 25 loci.

When determining the local level, the methods typically comprise a step of amplification of the gDNA fragments using a primer (e.g. a second primer as described in detail below in the section “Amplification”) specific for the locus of interest. If more than locus is of interest, said amplification step typically uses a mixture of primers comprising at least one (second) primer specific for each of the loci of interest.

The methods disclosed herein comprises a step of randomly selecting in the range of 100 to 100,000 tagged gDNA fragments per sample for sequencing. It is not a requirement that the exact number of gDNA fragments per sample is known. The samples are simply randomly selected and thus—in theory—a roughly equal number of gDNA samples per sample are sequenced. In practice it is preferred that n times 100 to 100,000 tagged gDNA fragments are sequenced, where n is the number of samples.

In some embodiments, the number of gDNA fragments selected in step i) depends on the number of loci of interest. Thus, in some embodiments, n times p times 10 to 100,000 tagged gDNA fragments are selected and sequences, such as n times p times 100 to 100,000, for example at the most n times p times 50,000, such as at the most n times p times 20,000, for example in the range of n times p times 10 to 50,000, such as in the range of n times p times 100 to 20,000 tagged gDNA fragments are selected for sequencing, wherein n is the number of samples provided in step a. and p is the number of loci of interest.

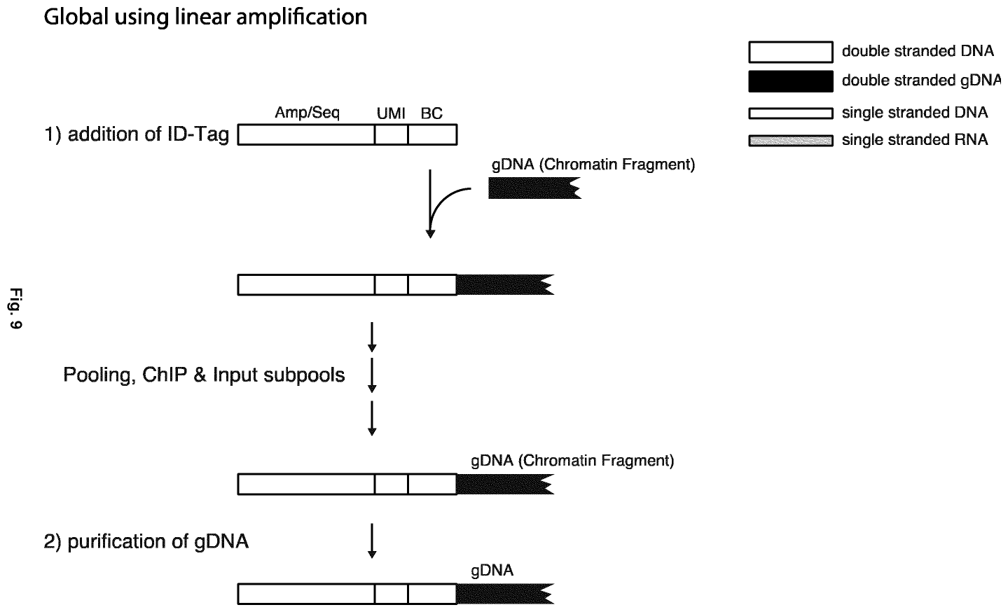

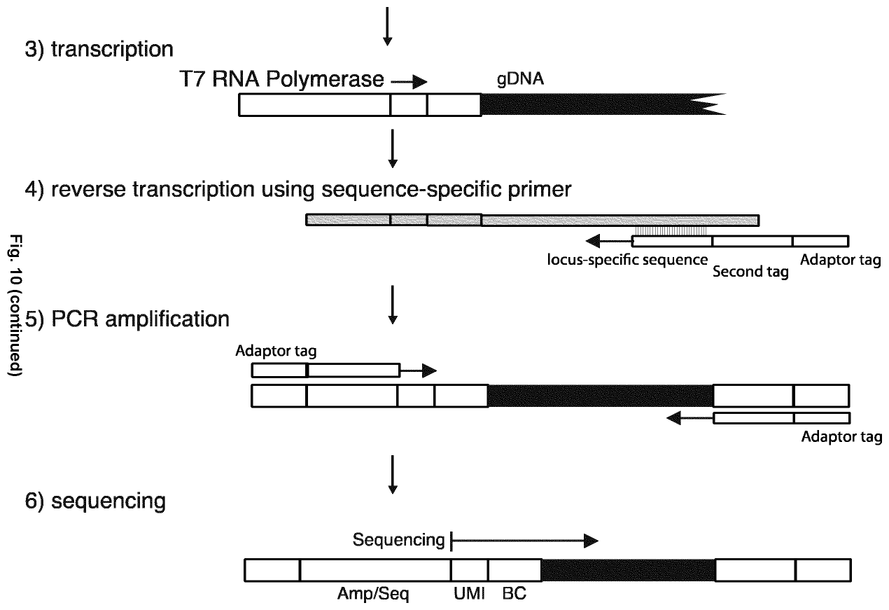

In embodiments of the invention, wherein local levels are determined, said gDNA fragments are selected from gDNA fragments within the chromatin modification sub-pools and input sub-pool, which can be mapped to one or more loci of interest. Frequently, the methods disclosed herein comprise a step of amplification of gDNA fragments from the chromatin modification sub-pools and input sub-pool. In embodiment of the invention, where the methods are for determining the local levels of a plurality of chromatin modifications, said step of amplification may be designed such that in principle only gDNA fragments from said one or more loci of interest are amplified. This may for example be achieved by tagging a random fraction of the gDNA fragments with an ID-tag comprising an amplification sequence. in any of the manners described below. The amplification may then be made with a primer recognising said amplification sequence, and a primer or mixture of primers specific for the loci of interest. Useful methods for tagging and amplification of gDNA fragments for assessing local levels of chromatin modification(s) are provided in FIGS. 8 and 10. The skilled person will be able to adapt the principles disclosed in those figures to other useful tagging and amplification methods.

### Sample

The methods of the invention allow for assessing the global levels of a plurality of chromatin modifications in a plurality of samples comprising chromatin.

Said samples comprising chromatin are preferably samples prepared from or comprising a cell population comprising a plurality of cells.

The methods allows for simultaneous testing of a plurality of samples. Thus, the methods may in particular comprise providing a plurality of test samples, such as at least 15, such as at least 25, for example at least at least 50, such as at least 75, for example in the range of 15 to 1000, such as in the range of 15 to 500, for example in the range of 25 to 1000, such as in the range of 25 to 500 different test samples comprising chromatin. The test samples are preferably provided in a manner, so that they are physically separated from each other.

One advantage of the methods of the invention is that the methods allow for accurate quantification of levels of multiple chromatin modifications in a plurality of different samples. Thus a large number of samples can be analysed in parallel. Accordingly, in some embodiments, at least 75, preferably at least 85, for example in the range of 75 to 1000, such as in the range of 75 to 500, for example providing in the range of 85 to 1000, such as in the range of 85 to 500 different test samples comprising chromatin are provided.

The samples usually comprise a cell population or the samples are prepared by purification or partial purification of chromatin from a cell population, wherein the cell population comprises a plurality of cells. The methods are particularly useful for assessing the effects of various treatments of cells on global levels of chromatin modifications.

Thus, the cell population preferably comprise a plurality of cells, which have all been treated in the same manner.

The cells may be any cells. In one embodiment, the cells are cultivated cells, however, the cells may also be obtained directly from single-celled or multicellular organisms, e.g. animals. The cells may for example be selected from the group consisting of transformed cell lines; primary cell lines, such as patient derived cell lines; cancer cell lines; iPS cells; adherent cells; suspension cells; 3D cell cultures; cells of engineered tissues and cell of organoids.

The cells comprised in or used for preparation of the test samples may have been cultivated by any useful method. For example, the cells may have been grown in suspension, grown as adherent cells, grown in 3D cultures or grown in aggregates e.g. organoids, gastruloids. Useful methods and media for cell cultivation are well known to the skilled person.

Prior to preparation of the sample, the cells may optionally be fixed, e.g. by incubation in formaldehyde. The cells may however also be native.

In general, in order to obtain an accurate assessment of chromatin modification levels, the sample must comprise or be prepared from a cell population comprising a plurality of cells. In particular, it is preferred that the sample is prepared from sufficient cells to allow preparation of input sub-pools and test sub-pools, where each chromatin fragment in principle is represented in each of said sub-pools. It follows, that a plurality of cells are required, because if chromatin from a single cell is divided into sub-pools, each of these will be different. Thus, the skilled person will appreciate that accurate assessment of chromatin modification levels cannot be made on a single cell level for several reasons.

Thus, in one embodiment, the cell population comprises at least 100 cells, preferably at least 500 cells, even more preferably at least 1000 cells, for example in the range of 10 to 100,000 cells, such as in the range of 100 to 100,000 cells, for example in the range of 1000 to 100,000 cells.

In one embodiment of the method, each sample comprises in the range of 10 to 100000 cells, such as in the range of 100 to 10000 cells or is purified/partly purified from aforementioned number of cells.

In preferred embodiments, the invention relates to assessing the effect of a plurality of treatments on the global level of a plurality of chromatin modifications. Said treatment may for example be incubation with different test compounds or combinations thereof. In such embodiments the methods are useful for assessing the effect of various test compounds on chromatin modification levels. This information can be used to evaluate the toxicity of compounds or combination of compounds.

Thus, the invention also provide methods of determining the influence of test compounds on the global or local levels of chromatin modifications, said method comprising the steps of
a. Providing one or more test compounds;
b. Cultivating a plurality of cells in the presence of said test compounds or combinations thereof, wherein cells cultivated in the presence of different test compounds or combinations thereof are physically separated from each other, and wherein cells cultivated in the presence of a given test compound or combination thereof is a cell population;
c. Performing the method of assessing the levels of a plurality of chromatin modifications in parallel in a plurality of samples according to the methods of the invention, wherein each of a plurality of test samples comprises chromatin from different cell populations

Whereas the cell populations cultivated in the presence of different test compounds or combinations thereof, it is comprised within the methods, that samples may be prepared in replicas, e.g. in duplicates or triplicates, and thus it is comprised within the invention that more than one, such as in the range of 1 to 10 cell populations may have been cultivated in the presence of the same test compound or combination of test compounds.

In embodiments of the invention, where the methods are used for assessing the effects of various treatments on global levels of chromatin, the methods may involve cultivating the same kind of cells with or without different kinds of treatments, and comparing the global levels of chromatin modifications in such cells. This will allow an assessment of the effect of the different treatments.

In preferred embodiments, said treatment may be incubation with different test compounds or combinations thereof. In such embodiments the methods are useful for assessing the effect of various test compounds on chromatin modification levels. This information can be used to evaluate the toxicity of compounds or combination of compounds.

In the same embodiments, the methods are useful in combination with a plurality of treatments that inhibiting or depleting enzyme involved in generating or removing chromatin modifications, for assessing the interdependence of a plurality of chromatin modifications (network analysis).

### Fragmentation of Chromatin

One aspect of the method relates to the fragmenting (fragmentation) of chromatin.

If the samples contain intact cells, the methods may comprise a step of disrupting the cellular membranes (e.g. by lysis), whereby native chromatin becomes accessible. Said chromatin may optionally be further purified or partially purified.

Chromatin of each sample may then be fragmented into chromatin fragments, wherein each chromatin fragment comprises a double-stranded genomic DNA (gDNA) fragment and optionally associated proteins. Said associated proteins may be histones and optionally other proteins.

Fragmentation of chromatin can be achieved by a variety of methods. In one embodiment of the method, said chromatin is fragmented by incubation with one or more enzymes catalysing fragmentation of chromatin. One or more of said enzymes are selected from the group consisting of nucleases, such as micrococcal nuclease (MNase); sequence-specific restriction enzymes or a mix thereof. In another embodiment the method of fragmentation of said chromatin is by mechanical shearing, such as by sonication, such as nebulization, such as focused acoustic shearing.

Chromatin may be fragmented into fragments in order to obtain an optimal size allowing for identification of a particular chromatin modification. In one preferred embodiment the size of the chromatin fragment is chosen such that the majority of fragments generated contain either zero or one instances of the chromatin modification, but not two or more.

In one embodiment, the size of the chromatin fragments is chosen, such that the majority of fragments generated (e.g. at least 50%, such as at least 60%, for example at least 70%, such as at least 80% of fragments generated) contain one, and not more than one nucleosome unit, which comprises two copies of each core histone. This may in particular be the case in relation to methods, wherein one or more chromatin modifications assessed are histone PTMs. In this embodiment, the majority of fragment are expected to contain either zero, one, or two instances of the chromatin modification, but not three or more.

In one embodiment, the optimal chromatin fragment size is in the range between 140 bp and 200 bp, which correspond to the size of a nucleosome. This may in particular be the case, when one or more chromatin modifications assessed are histone PTMs. In another embodiment the optimal size for corresponds to the expected minimal distance of the modification on DNA. This may in particular be the case, when the chromatin modifications assessed are modification of nucleotides. In embodiments, where one or more chromatin modifications are the presence of proteins binding chromatin, the optimal size corresponds to the expected minimal distance of the protein binding sites but minimally e.g. 1-2 times the length of the expected footprint of the protein on DNA.

In one embodiment said chromatin fragments are fragmented into fragments ranging from 100 bp to 10 kb in size.

### Tagging

As described above, the methods of the invention comprise fragmenting chromatin into chromatin fragments. Each chromatin fragment comprise a gDNA fragment and optionally associated proteins. The methods disclosed herein may further comprise a step tagging of the gDNA fragments with one or more tags.

The term “tagging” as used herein refers to adding (e.g. by ligation) a gDNA n fragment with a short sequence of nucleotides, e.g., DNA, RNA, or nucleotide analogues. Tagging may be performed on gDNA in complex with proteins (e.g, on chromatin fragments) or on purified or otherwise pretreated gDNA.

Tagging may be performed for various reasons. However, the ID-tag is primarily added in order to allow attributing a given gDNA fragment to a given sample by means of sequencing. Thus, tagging may for example be an aid in identification of sample origin. Tagging may also for example be an aid in identification of unique molecules, and/or it may for example enable mapping and quantification of global levels of chromatin modifications.

Tagging may also be performed for enabling amplification, selection, extension, modification of the gDNA fragment, for example enabling sequencing with a specific sequencing platform.

A tag may combine several of the functions described above.

A ‘tag’ as defined here is typically an oligonucleotide with a specific sequence. The oligonucleotide may be single or double stranded or partially single stranded and partially double stranded. The tag oligonucleotide is in general DNA, but it may also comprise other types of nucleotides.

### ID-Tag

The chromatin of each sample is fragmented into chromatin fragments, wherein each chromatin fragment comprises a double-stranded genomic DNA (gDNA) fragment (optionally with single stranded overhangs) and optionally associated proteins. Then, gDNA fragments of each sample may be tagged with a ID-tag.

The ID-tag may be an oligonucleotide, preferably a double stranded oligonucleotide. The ID-tag comprises a barcode sequence and optionally additional sequences. The barcode sequence of the ID-tag may also be referred to as “first barcode sequence” herein. The first barcode sequence is a unique sequence identifying each sample. In other words, the gDNA fragments of one sample are tagged with the same first barcode sequence, whereas gDNA fragments of different samples are tagged with different first barcode sequences. In that manner, the ID-tag can be used to identify from which sample a particular gDNA fragment is derived.

It is noted that whereas it is possible that essentially all gDNA fragments within a sample are tagged, it is also comprised within the invention, that only a fraction of the gDNA fragments are tagged. By example, only 1%, 0.1%, 0.01% or less of the gDNA fragments may be tagged. It is preferred that if only a fraction of the gDNA fragments are tagged with the ID-tag, said fragments are randomly selected.

The barcode sequence may be in the range of 4 to 40 nucleotides, for example in the range of 4 to 20 nucleotides, such as in the range of 6 to 16 nucleotides. In principle, the barcode sequences may be any sequence, provided that the barcode sequences are different for each sample. In some embodiments it may be preferred that the barcode sequence does not form secondary structures to any significant degree.

In addition to the first barcode sequence, the ID-tag may contain additional sequences. For example, the ID-tag may comprise a random DNA sequence acting as a unique molecular identifier, also referred to as a UMI sequence herein. Thus, in principle each UMI sequence is different. The UMI may comprises a random sequence of in the range of 4 to 20 nucleotides, for example in the range of 6 to 16 nucleotides.

In such embodiments, the tagged gDNA fragments within one sample will be tagged with the same first barcode sequence, but with different UMI sequences.

In one embodiment the ID-tag may comprise additional sequences in addition to the first barcode sequence and the UMI sequence. Said additional sequences may facilite the tagging process, i.e. they may aid in attaching the tag to the gDNA fragment. For example the additional sequence may be s single stranded overhang, which can anneal to single stranded ends on the gDNA fragments. Single stranded ends on gDNA fragments of a specific sequence may for example be generated if the fragments are produced by the aid of restriction enzymes. Thus, the additional sequence may be a single stranded overhang, which is complementary to the single stranded overhang produced by a restriction enzyme.

The ID-tag may also comprise additional sequence(s) useful for amplification and/or sequencing. Such sequences are also referred to as “amplification sequences” and “adaptor sequences”, respectively herein. The amplification sequences may be chosen to be useful as primer binding sites. Thus, the amplification sequence may comprise or consist of any sequence to which a primer can anneal. The skilled person is able to design a sequence useful as primer binding sites. The amplification sequence may also comprise or consist of a polymerase promoter, such as an RNA polymerase promoter.

The adaptor sequences are in general chosen in accordance with the sequencing methodology to be used for the particular method.

Thus, some sequencing methods comprise a step of amplification, e.g. a PCR, clonal PCR or a compartmentalised PCR as described in more detail below. In such embodiments, the ID-tag may comprise an adaptor sequence comprising a sequence, which can anneal to a primer.

Some sequencing methods comprise a step of immobilisation of gDNA fragments, typically by hybridisation to one or more immobilised oligonucleotides. In such embodiments the ID-tag may comprise an adaptor sequence, which is a single stranded and capable of annealing to at least one of said immobilised oligonucleotides.

In embodiments of the invention, wherein the methods disclosed herein comprises a step of ligating a second tag to the gDNA fragments, the ID-tag may be designed so that it cannot be ligated to other sequences, once it is ligated to the gDNA fragment. In other words, the ID-tag may comprise a non-ligatable end.

The ID-tag may be a double-stranded oligonucleotide optionally containing one or two single stranded overhang(s). One strand of the ID-tag preferably contains at the most 100 nucleotides, such as at the most 75 nucleotides, for example at the most 50 nucleotides. In particular, the ID-tag may consist of an oligonucleotide consisting of in the range of 6 to 100 nucleotides or nucleotide base pairs, such as in the range of 6 to nucleotides or nucleotide base pairs, for example in the range of 6 to 50 nucleotides or nucleotide base pairs.

### Second Tag

The methods described herein comprise steps in which chromatin fragments carrying a specific chromatin modification are isolated from a pool or sub-pool of chromatin fragments by adsorption to a specific antibody (“immunoprecipitation”) are obtained and optionally further processed.

For example, the chromatin fragments or the gDNA fragment part thereof may be purified, amplified and/or otherwise prepared in a manner that allow subsequent sequencing.

One step in such procedure may be tagging of the chromatin fragment (typically the gDNA part of the chromatin fragment) with a second tag, in order to for example assist selection, amplification or identification in subsequent steps.

Herein a composition comprising all gDNA fragments of all chromatin fragments binding a specific antibody are also referred to as “chromatin modification sub-pool”. Thus, a “chromatin modification sub-pool” in principle contain gDNA fragment only from such chromatin fragments, which contain the chromatin modification recognised by the specific antibody. In order to identify which “chromatin modification sub-pool” a given gDNA fragment is derived from, the second-tag may comprise a barcode sequence (subsequently referred to as “second barcode sequence” or “second barcode”).

The second barcode sequence is a unique sequence identifying gDNA fragments from each chromatin modification sub-pool. In other words, the gDNA fragments of one chromatin modification sub-pool are tagged with the same second barcode sequence, whereas gDNA fragments of different chromatin modification sub-pools are tagged with different second barcode sequences.

In that manner, a gDNA may be tagged with a ID-tag, which can be used to identify from which sample a particular gDNA fragment is derived and a second tag, which can be used to identify from which chromatin modification pool a particular gDNA fragment is derived.

It is noted that whereas it is possible that essentially all gDNA fragments within a chromatin modification sub-pool are tagged with the second-tag, it is also comprised within the invention, that only a fraction of the gDNA fragments are tagged. It is thus also comprised within the invention that only a fraction of gDNA fragments are tagged with a combination of one ID-tag and one second-tag.

The second tag may be an oligonucleotide, preferably a double stranded oligonucleotide.

The second tag may be attached to the gDNA fragment by any useful means. In one embodiment, the second tag may be ligated onto the gDNA fragment, e.g. as a double stranded oligonucleotide.

The methods described herein may comprise a step of amplifying gDNA fragments on the chromatin modification sub-pool. In such embodiments, the second tag may be added during the amplification process. For example, the second tag may be part of a primer used for amplification.

Thus, in one embodiment, a single-stranded oligonucleotide representing part of the second tag may be ligated onto the gDNA fragment. Subsequently, a oligonucleotide representing a complementary part of the second tag may be hybridized to the first part, now forming a double stranded oligonucleotide.

In another embodiment, a single-stranded oligonucleotide representing part of the second tag may be ligated onto the gDNA fragment. The second tag is converted to a double stranded oligo by virtue of DNA polymerase activity synthesizing a complementary strand during a subsequent amplification step.

In another embodiment, a single-stranded oligonucleotide representing part of the second tag may be hybridized onto the gDNA fragment by virtue of a sequence in the second tag being complementary to the gDNA sequence

In another embodiment, a single-stranded oligonucleotide representing part of the second tag may be ligated onto single-stranded RNA that is transcribed and from the gDNA fragment in an amplification step. Said amplification step may for example use a primer binding to amplification sequences in the ID-tag. The second ID-tag may then be converted to a double stranded oligo by virtue of DNA polymerase activity synthesizing a complementary strand during a subsequent amplification step.

In another embodiment, a single-stranded oligonucleotide representing part of the second tag may be hybridized onto single-stranded RNA that is transcribed and from the gDNA in an amplification step. The second tag may be converted to a double stranded oligo by virtue of DNA polymerase activity synthesizing a complementary strand during a subsequent amplification step.

It is noted that whereas it is possible that essentially all gDNA fragments within a chromatin modification pool are tagged, it is also comprised within the invention, that only a fraction of the gDNA fragments are tagged.

The second-tag may comprise a second barcode sequence and optionally additional sequences useful for ligation to, hybridization to, or amplification of a gDNA fragments. Such sequences may also be referred to as “amplification sequences”.

The second barcode sequence may be in the range of 4 to 20 nucleotides, for example in the range of 6 to 16 nucleotides.

In addition to the second barcode sequence, the second tag may contain additional sequences. Said additional sequences may facilitate the tagging process, i.e. they may aid in attaching the tag to the gDNA fragment. For example the additional sequence may be s single stranded overhang, which can anneal to single stranded ends on the gDNA fragments or on gDNA fragments tagged with a ID-tag. Single stranded ends on gDNA fragments of a specific sequence may for example be generated if the fragments are produced by the aid of restriction enzymes or if the first ID-tag contains a single stranded overhang. Thus, the additional sequence may be a single stranded overhang, which is complementary to the single stranded overhang produce by a restriction enzyme.

In some embodiments it is preferred that the second tag is added to the opposite end of the gDNA fragment compared to the ID-tag. Thus frequently, if the second tag is to be ligated onto the gDNA fragments, the ID-tag may be designed so that it cannot be ligated to other sequences, once it is ligated to the gDNA fragment.

The second ID-tag may also comprise additional sequence(s) useful for amplification and/or sequencing. Such sequences are also referred to as “amplification sequences” and “adaptor sequences”, respectively herein. The adaptor sequences are in general chosen in accordance with the sequencing methodology to be used for the particular method.

Thus, some sequencing methods comprise a step of amplification, e.g. a PCR, a clonal PCR or a compartmentalised PCR as described in more detail below. In such embodiments, the second tag may comprise an adaptor sequence comprising a sequence, which can anneal to a primer. Such sequence is also referred to as “primer docking sequence”. If both the first ID-tag and the second ID-tag comprise a primer docking sequence, they may be chosen such that the sequence between said primer docking sequences can be amplified using a primer pair annealing said primer docking sequences.

Some sequencing methods comprise a step of immobilisation of gDNA fragments, typically by hybridisation to one or more immobilised oligonucleotides. In such embodiments the second tag may comprises an adaptor sequence, which is single stranded and capable of annealing to at least one of the immobilised oligonucleotides. Such sequence is also referred to as “hybridisation sequence”. If both the first ID-tag and the second tag comprises hybridisation sequences, those may be the same or different sequences. If they are different sequences, they can anneal to different immobilised oligonucleotides at the same time and thereby being useful for bridge amplification.

The second tag may be a double-stranded oligonucleotide optionally containing one or two single stranded overhang(s). One strand of the second tag preferably contains at the most 100 nucleotides, such as at the most 75 nucleotides, for example at the most 50 nucleotides. In particular, the second ID-tag may consist of an oligonucleotide consisting of in the range of 6 to 100 nucleotides or nucleotide base pairs, such as in the range of 6 to 75 nucleotides or nucleotide base pairs, for example in the range of 6 to 50 nucleotides or nucleotide base pairs.

### Tagging Methods

In one embodiment the method constitutes tagging the gDNA fragments with a first ID-tag and/or a second tag and/or an additional tag. Tagging typically comprises ligating said first ID-tag and/or a second tag to the gDNA fragments. Alternatively, tagging may be done by tagmentation or tags may be added as part of an amplification process. Tagging may also be done as part of an amplification process. For example tagging can be achieved using primers comprising a 3′ primer annealing sequence and a 5′ tag sequence for amplification. Thus, the tag may be added to the original gDNA fragment and/or to copies of the gDNA fragment created by amplification. For the sake of simplicity, the term “gDNA fragments” as used herein may collectively refere to the original gDNA fragments and copies thereof.

It is also comprised within the invention that a tag is added in multiple steps, for example by any combination of the methods described above. For example, a part of a tag can be added by ligation, whereas the rest can be added during amplification as part of a primer.

In one embodiment the method constitutes tagging the gDNA fragments with an ID-tag, and a second tag, which may be any of the ID-tags and second tags described herein above.

In one embodiment the method constitutes tagging the gDNA fragments with an ID-tag. In the course of the method, the ID-tag and the gDNA sequence of each tagged chromatin fragment may be transcribed into RNA, with each tagged chromatin fragment giving rise to one or more RNA molecules with the same sequence. The second tag may then be ligated or hybridized as an oligonucleotide as specified above to the RNA copy of the tagged chromatin fragment, not the original gDNA fragment.

The tagging may be done by any means useful for attaching an oligonucleotide to a gDNA fragment. Frequently, the tagging will be performed by ligation, for example using adaptor ligation using DNA ligase, such as using a DNA ligase, including but not limited to T4 DNA ligase, T3 DNA ligase, T7 ligase. In another embodiment the said tagging is performed using adaptor ligation using a transposon enzyme, such as using Tn5 transposase, Sleeping Beauty transposase, Tn7 transposase.

Examples of useful tagging methods are illustrated in FIGS. 7 to 10. The skilled person will be able to vary the specific methods used based on the common general knowledge and the overall guidance given in these figures.

### Immunoprecipitation

The methods of the invention comprises a step of isolating chromatin fragments binding specific antibodies. Said step may also be referred to as “immunoprecipitation” or “ChIP”.

Typically, the immunoprecipation is performed on a combined pool of chromatin fragments, wherein at least some chromatin fragments from each sample have been tagged with a first ID-tag. Thus, the method may comprise a step of combining chromatin fragments tagged with a first ID-tag, thereby generating a pool of chromatin fragments carrying different first barcode sequences. As noted above, it is not required that all chromatin fragments are tagged with a first ID-tag, and thus the pool of chromatin fragments may comprise both tagged and un-tagged chromatin fragments.

Once the pool of chromatin fragments is generated, said pool may be used for immunoprecipitation. Typically, the pool of chromatin fragments is divided into random sub-pools, wherein one sub-pool is an input sub-pool (“Input”) and the other sub-pools are test sub-pools. Each sub-pool contain a representative mix of chromatin from all the samples.

It is noted that in order to prepare a plurality of test sub-pools and an input sub-pool, which largely contains the same chromatin fragments, the sample must be prepared from a plurality of cells as described herein above in the section “Sample”. Thus, if a sample were only prepared from one single cell, the sample cannot be divided into several sub-pools before immunoprecipitation. If one were to divide such a sample into several sub-pools, each of these sub-pools would necessarily have to be different from each other, because the sample in principle would contain only one of each chromatin fragment. Thus, it is preferred that the sample is prepared from a cell population comprising at least a number of cells equal to the number of sub-pools. In other words, preferably the sample is prepared from a cell population comprising at least a number of cells equal to the number of antibodies provided plus 1, even more preferably at least 100 or at least 500 cells as described herein elsewhere.

Each immunoprecipation is performed by incubating an antibody specifically binding a chromatin modification with the pool of tagged chromatin fragments or with a sub-pool comprising a random fraction thereof (also referred to as a “test sub-pool”). The methods comprise use of a plurality of different antibodies, for example at least 5 different antibodies, such as at least 10 different antibodies, for example at least 15 different antibodies, such as in the range of 5 to 100 different antibodies, for example in the range of 5 to 50 different antibodies, such as in the range of 10 to 100 different antibodies, for example in the range of 10 to 50 different antibodies.

As noted above, in some embodiments it is preferred that at some of the chromatin modifications are histone PTMs. In such embodiments, it is preferred that a plurality of antibodies binding different histone PTMs, for example at least 5 different antibodies binding different histone PTMs, such as at least 10 different antibodies binding different histone PTMs, for example at least 15 different antibodies binding different histone PTMs, such as in the range of 5 to 100 different antibodies binding different histone PTMs, for example in the range of 5 to 50 different antibodies binding different histone PTMs, such as in the range of 10 to 100 different antibodies binding different histone PTMs, for example in the range of 10 to 50 different antibodies binding different histone PTMs are provided and used for immunoprecipitation.

Typically, each antibody is incubated with a test sub-pool. Accordingly, the pool of chromatin fragments is typically randomly divided into sufficient random sub-pools, so that there is at least one test sub-pool per antibody. For example, the pool of chromatin fragments may randomly be divided into X+Y random sub-pools, wherein X is the number of different antibodies for immunoprecipitation and Y is the number of desired additional sub-pools comprising e.g. a input sub-pool. Thus Y may be 1 to 3, such as 1.

The immunoprecipitation reactions may be performed in parallel. Each immunoprecipitation reaction in general constitutes a procedure in which an antibody that specifically binds a chromatin modification (e.g. a PTM) is incubated with a test sub-pool followed by separation of the antibody and any chromatin fragments binding thereto from the remainder of the test sub-pool. The different antibody incubation reactions are normally physically separated from each other. To allow easy separation, the antibody may be immobilised onto a solid support. Examples of such solid support include, but are not limited to beads, surfaces of a container, such as a microwell or a microfluidic surface.

Once separated from the test-pool, the chromatin fragments binding a particular antibody may be recovered. Any composition comprising the gDNA fragment of said chromatin fragments is referred to as “chromatin modification sub-pool”, because each of these sub-pools in principle only comprises gDNA fragment from chromatin fragments comprising the specific chromatin modification recognised by the antibody used for immunoprecipitation. All or a fraction of these chromatin fragments may be purified, selected, tagged with second tags and/or amplified as described above.

In some embodiments of the invention, the chromatin modification sub-pools are maintained physically separately. In such embodiments, roughly the same number of gDNA fragments are randomly selected from each chromatin modification sub-pool.

In other embodiments gDNA fragments from several or all of the chromatin modification sub-pools are mixed into a combined pool. This is in particular the case in embodiments, where gDNA fragments are tagged with a second tag comprising a second barcode, which can be used to identify from which chromatin modification sub-pool a given gDNA fragment is derived.

In such embodiments, it is preferred that in the range of n times m times 100 to 100,000 tagged gDNA fragments are selected from said combined pool.

A vast number of suitable antibodies exist that specifically binds chromatin modifications, and examples of useful antibodies are given below.

### Antibodies

The antibodies used for immunoprecipitation may be any antibody specifically binding a chromatin modification of interest. Numerous antibodies binding various chromatin modifications are commercially available. Alternatively, the skilled person is aware how to generate antibodies specifically binding an epitope of interest.

The antibody may be any antibody, such as a monoclonal antibody, a polyclonal antibody, an immunoglobulin, or an antigen binding fragment of an immunoglobulin. Thus, the antibody may be an antibody of classes IgG, IgM, IgA, IgD or IgE, or fragments or derivatives thereof, including Fab, F(ab′)2, or Fd fragments. The antibody may also be single chain antibodies, domain antibodies, diabodies, bispecific antibodies, bifunctional antibodies and derivatives thereof. Typically, an antibody will comprise a variable region comprising 3 CDRs.

Each antibody to be used with the methods of the invention may thus bind an epitope comprising or consisting of any of the chromatin modifications described herein above. In particular, one or more of the antibodies to be used with the methods of the invention may bind an epitope comprising or consisting of any of the posttranslational modifications (PTMs) described herein above.

### Amplification

The methods disclosed herein frequently comprise a step of amplification of gDNA fragments of the chromatin modification sub-pool. The process of amplification entails creation of new DNA molecules with the same sequence as the original tagged gDNA molecule or a part thereof.

The gDNA fragments of each chromatin modification sub-pool may be amplified separately. Alternatively, one or more chromatin modification sub-pools may be combined before amplification. The latter is usually only done, if the gDNA fragments of each chromatin modification sub-pool have been tagged with a second tag comprising a second barcode allowing to identify from which chromatin modification sub-pool a tagged gDNA fragment is derived.

While the product of amplification typically is a plurality of DNA molecules carrying the entire or part of the original tagged gDNA sequence, an intermediate step may entail generation of RNA molecule copies transcribed from the original gDNA sequence that then are reverse transcribed to create DNA molecules carrying the entire or part of the original gDNA sequence.

The amplification is in general performed on tagged gDNA fragments of the chromatin fragments obtained after immunoprecipitation. The amplification may be performed before or after tagging with the second tag. Thus, as described above, the second tag may not be added to the original gDNA fragment, but instead to a DNA or RNA copy of said gDNA fragment produced via amplification. As a control, the gDNA fragments of the input sub-pool (“Input”) may be amplified and otherwise treated in the same manner as gDNA fragments from the chromatin modification sub-pools. It is generally preferred that any steps of amplification are performed in the same manner for all chromatin modification sub-pools and the input sub-pool.

The amplification may be performed by any useful means, e.g. by polymerase chain reaction (PCR), linear amplification via an RNA intermediate and/or reverse transcription using adequate primers.

Primers are single stranded oligonucleotides. As described above, the first ID-tag, and/or the second tag may comprise amplification sequences useful for amplification. When the methods disclosed herein comprise a step of amplification, the ID-tag usually comprises an amplification sequence. The amplification step can then be performed with the aid of a primer capable of annealing to said amplification sequence.

In some embodiments, the amplification comprises a step of transcription of the gDNA fragments. In such embodiments, the ID-tag typically comprises an amplification sequence comprising an RNA polymerase promoter. This will allow an RNA polymerase to transcribe the gDNA fragment. In such embodiments, the methods may further comprise a step of reverse transcription. Examples of such methods are shown as steps 3 to 5 in FIG. 9 and steps 3 to 4 in FIG. 10.

In some embodiments, the amplification comprise a step of PCR. In such embodiments, the ID-tag usually comprises an amplification sequence to which a primer can anneal. Thus, the first primer may contain a sequence, which is identical or complementary to an amplification sequence of the ID-tag. In addition, the first primer may contain additional sequences, e.g. adaptor sequences (as described above). Said additional sequences preferably consist of no more than 100, such as no more than 50, for example no more than 25 nucleotides.

The second primer used for the amplification may be a primer annealing to an amplification sequence in the second tag (or to a sequence complementary thereto). The second primer used for amplification may also be degenerate primers, universal primers or a collection of random primers. In the aforementioned cases, said primers are preferably designed to anneal to many different locations in the genome allowing global ampliifcation of gDNA fragments covering the whole genome or at least as much of the genome as possible. Said degenerate primers, universal primers or random primers are typically rather short, e.g. in the range of 5 to 10 nucleotides, such as in the range of 5 to 7 nucleotides enhancing the chance of amplification of gDNA fragments largely covering the genome. This is in particular the case, if the method is for assessing global levels of the chromatin modifications.

The second primer used for amplification may also be a primer specific for one or more loci of interest. This is in particular the case, if the method is for assessing local levels of the chromatin modifications.

Thus, the second primer may contain a sequence, which is identical or complementary to an amplification sequence of the second tag or to sequence(s) specific for one or more loci of interest, or a degenerative sequence. In addition, the second primer may contain additional sequences, e.g. parts of the second tag and/or adaptor sequences (as described above). Said additional sequences preferably consist of no more than 100, such as no more than 50, for example no more than 25 nucleotides.

For example, the first primer may be a primer binding:
An amplification sequence within the ID-tag
An amplification sequence within the second tag
A genomic sequence
   The first primer may also bind degenerate or random sequence(s).

Similarly, the second primer may be a primer binding:
An amplification sequence within the ID-tag
An amplification sequence within the second tag
A genomic sequence
   The second primer may also bind a degenerate or random sequence(s).

For amplification by PCR the first and the second primer are chosen so that they together are capable of priming amplification of sequences positioned in between the complementary sequence in the tagged gDNA fragments.

The methods may thus comprise a step of amplification of at least a part of the gDNA fragments, e.g. of the single tagged or double tagged gDNA fragments to obtain copies of the gDNA fragments, wherein said copies preferably contain at least the parts of the original tagged gDNA fragments described below.

If the primers used anneal to amplification sequences in the tags, the amplification may be performed indiscrimative of the gDNA sequence itself.

In one embodiment, the amplification does however not rely on a universal sequence present in a tag added to the gDNA, but on one or more specific genomic sequences. Said specific genomic sequences may be selected by using adequate primers comprising complementary sequences to the specific genomic sequences. Thereby only those fragments comprising said sequences are amplified.

Typically, the amplification is performed prior to sequencing.

Whereas amplification may be useful, too much amplification generates a risk that the same sequence is sequenced more than once. Thus, if there are many copies of the same gDNA fragment, the same gDNA fragment may be randomly selected for sequencing more than once. Accordingly, it is preferred that PCR amplification comprises at the most 20 cycles, such as in the range of 5 to 20 cycles, for example in the range of 5 to 15 cycles.

If the methods comprise a step of amplification, sequencing is normally done on said amplified fragments. Thus, in such embodiments it is copies of gDNA fragments, which are sequenced rather than the original gDNA fragments themselves. It is noted that said copies may not be exact copies of the original gDNA fragments, but may lack parts of the original gDNA fragments and/or contain additional sequences introduced as part of the primers. In order to simplify matters, the term “gDNA fragments” may refer both to the original gDNA fragments as well as to copies thereof. Copies of the original gDNA fragments are only considered to be “gDNA fragments” if they contain at least the first barcode sequences as well as the UMI sequence and/or at least a part of the gDNA sequence, which is sufficiently long to establish whether it is unique (i.e. at least 10, such as at least 15 nucleotides long). Preferably, copies of the original gDNA fragments are only considered to be “gDNA fragments” if they contain at least the first barcode sequences as well as the gDNA sequence. In embodiments, where the ID-tag contains a UMI, copies of the original gDNA fragments are preferably only considered to be “gDNA fragments” if they contain at least the first barcode sequence and the UMI sequence, and more preferably only if they contain at least the first barcode sequences, the UMI sequence and at least a part of the gDNA sequence, which is sufficiently long to establish whether it is unique (i.e. at least 10, such as at least 15 nucleotides long). In embodiments, wherein the second tag comprises a second barcode, copies of the original gDNA fragments are preferably only considered to be “gDNA fragments” if they contain at least the first barcode sequence and the second barcode as well as the UMI sequence and/or at least a part of the gDNA sequence, which is sufficiently long to establish whether it is unique (i.e. at least 10, such as at least 15 nucleotides long).

### Randomly Selecting gDNA Fragments

As described in detail above, chromatin modification sub-pools are prepared, wherein each chromatin modification sub-pool in principle only contains gDNA fragments from chromatin fragments comprising a certain chromatin modification. If the methods comprise a step of amplification as described above, said gDNA fragments selected for sequencing may be the original tagged gDNA fragments, or it may be copies of the original gDNA fragments or a mixture of both, wherein said copies may be gDNA fragments as described in the section “Amplification” above.

In parallel with preparation of the gDNA fragments of the test sub-pools, and the input sub-pool is subjected to the same treatment (e.g. tagging) expect that it is not subjected to immunoprecipitation. In that manner the input sub-pool can be used as control or reference.

In one embodiment, the methods comprise a step of tagging gDNA fragments of each chromatin modification sub-pool and the input sub-pool with a second tag, wherein gDNA fragments within one chromatin modification sub-pool/input sub-pool is tagged with a second tag comprising the same second barcode sequence, and wherein different second barcode sequences are used for each chromatin modification sub-pool or input sub-pool. This procedure is described in more detail above.

In such embodiments, the second barcode can be used to identify from which chromatin modification sub-pool a given gDNA fragment is derived.

In some embodiments, the methods comprise a step of pooling the tagged gDNA fragments from more than one chromatin modification sub-pool and the input sub-pool. If the methods comprise a step of amplification as described above, said pooling may be performed before or after said amplification. Thus, it may be the original tagged gDNA fragments, which are pooled, or it may be copies of the original tagged gDNA fragments or a mixture of both, wherein said copies may be gDNA fragments as described in the section “Amplification” above.

Preferably, the method may comprise a step generating a pool of gDNA fragments by pooling gDNA fragments from all chromatin modification sub-pools and from the input sub-pool. This may in particular be relevant in such embodiments of the invention, where gDNA fragments of each chromatin modification pool and the input sub-pool are tagged with a second ID-tag. In such embodiments the pool of gDNA fragments will comprise gDNA fragments tagged with a first ID-tag, which can be used to identify from which sample the gDNA fragment is derived and a second ID-tag, which can be used to identify which chromatin modification was associated with the gDNA fragment.

The methods then comprises a step of randomly selecting from said pool in the range of 100 to 100,000, such as in the range of 200 to 100,000, for example at the most 50,000, such as at the most 20,000, for example in the range of 200 to 50,000, for example in the range of 1000 to 50,000, such as in the range of 5000 to 20,000 tagged gDNA fragments per test sample. Thus, if n samples are analysed by the methods of the invention (i.e. if n test samples are provided in step a), then n times the aforementioned number of tagged gDNA fragments are randomly selected. It is not required that exactly the same amount of tagged gDNA fragments are provided for each test sample. It is sufficient that the tagged gDNA fragments are randomly selected, which ensures representation of the various test samples.

Thus, in other words, the methods may comprise a step of randomly selecting from said pool in the range of n times 100 to 100,000, such as in the range of n times 200 to 100,000, for example at the most n times 50,000, such as at the most n times 20,000, for example in the range of n times 200 to 50,000, for example in the range of n times 1000 to 50,000, such as in the range of n times 5000 to 20,000 tagged gDNA fragments, wherein n is the number of test samples provided in step a.

It is not required that an exact number of gDNA fragments are selected for sequencing. It is sufficient that an approximate number of gDNA fragments are selected. A trivial procedure for randomly selecting gDNA fragments for sequencing is to aliquot a volume comprising the expected number of molecules.

In some embodiments, the number of tagged gDNA fragments randomly selected for sequencing is dependent on the number of chromatin modification sub-pools. Accordingly, in one embodiment, the methods of the invention comprises randomly selecting from said pool in the range of n times m times 100 to 100,000, for example at the most n times m times 50,000, such as at the most n times m times 20,000, for example in the range of n times m times 1000 to 50,000, such as in the range of n times m times 5000 to 20,000 tagged gDNA fragments, wherein n is the number of test samples provided in step a., and m is the number of chromatin modification sub-pools and/or m is the number of antibodies provided in step e. Said selected fragments are then subjected to sequencing.

In one embodiment, the methods of the invention comprises randomly selecting from said pool in the range of n times m times 200 to 100,000, for example at the most n times m times 50,000, such as at the most n times m times 20,000, for example in the range of n times m times 200 to 50,000, for example in the range of n times m times 1000 to 50,000, such as in the range of n times m times 5000 to 20,000 tagged gDNA fragments per test sample per chromatin modification sub-pool, wherein n is the number of test samples provided in step a., and m is the number of chromatin modification sub-pools and/or m is the number of antibodies provided in step e. Said selected fragments are then subjected to sequencing.

Thus, if n samples are analysed by the methods of the invention (i.e. if n test samples are provided in step a), and m different antibodies are provided, then n times m times the aforementioned numbers of tagged gDNA fragments are randomly selected.

In other embodiments, the gDNA fragments of the various chromatin modification sub-pools are not combined in a pool. In such cases, aforementioned number of gDNA fragments are randomly selected from the chromatin modification sub-pools. This is frequently done in a manner so approximately the same number of gDNA fragments are selected for sequencing from each sub-pool. A trivial procedure for randomly selection molecules approximately the same number of gDNA fragments is to aliquot a volume comprising the expected number of molecules. For example, the same volume may be aliqouted from each chromatin modification sub-pool and the input sub-pool. However, since the methods typically comprises determining the fraction or percentage of each first barcode in each chromatin modification sub-pool, it is not a requirement that the same number of gDNA fragments are sequenced per chromatin modification sub-pool. Typically, at least 100, such as at least 200, for example at least 1000, such as at the most 100,000, for example at the most 50,000, such as at the most 10,000 gDNA fragments are randomly selected from each chromatin modification sub-pool. In particular, n times 100 to 100,000, for example at the most n times 50,000, such as at the most n times 20,000, for example in the range of n times 1000 to 50,000, such as in the range of n times 5000 to 20,000 tagged gDNA fragments are randomly selected from each chromatin modification sub-pool and sequenced, wherein n is the number of test samples provided in step a.

By way of example, the term “n times 100 to 100,000” means “n×100 to n×100,000” and the term “n times m times 100 to 100,000” means “(n×m×100) to (n×m×100,000)”.

### Sequencing

The randomly selected tagged gDNA fragments are then sequenced. It is also comprised within the invention that the steps of randomly selecting and sequence are combined and performed simultaneously. It is not required to sequence the entire gDNA fragments. In general however, at least the first barcode sequence is sequenced together with sufficient sequence to establish whether the gDNA fragment is a unique gDNA fragment. The sequencing step may comprise a step of amplification as described below. Said amplification may be performed in addition to any amplification performed prior to randomly selecting gDNA fragments for sequencing. In some embodiments only part of the tagged gDNA fragments are amplified, in which case only said amplified part or a fragment thereof is sequenced.

The minimum parts to be sequenced are described below.

In some embodiments of the invention, the first ID-tag comprises a UMI sequence as described above. When the first ID-tag comprises a UMI sequence, said UMI sequence may be used alone or in combination with gDNA sequences to establish whether the tagged gDNA fragment is a unique gDNA fragment. In such embodiments
at least the first barcode sequence and the UMI sequence or
at least the first barcode sequence and the UMI sequence and at least part of the gDNA sequence, such as the entire gDNA sequence is sequenced.

In embodiments, where the first ID-tag does not comprise a UMI sequence, the first barcode sequence as well as at least part of the gDNA sequence, such as the entire gDNA sequence is sequenced.

In some embodiments of the invention, the gDNA are tagged with both a first ID-tag and a second ID-tag. In such embodiments, at least the first barcode sequence and the second barcode sequence are sequenced along with the UMI sequence and/or at least part of, such as the entire the gDNA sequence.

In some embodiment the entire tagged gDNA fragments are sequenced.

The sequencing may be performed in by any useful method. For example, the sequencing may be done by direct single molecule sequencing (e.g. nanopore sequencing).

Preferably, the sequencing is done using massive parallel sequencing. Massive parallel sequencing is also known as next-generation sequencing (NGS) or second-generation sequencing. Massive parallel sequencing involves parallel sequencing of a large number of spatially separated DNA templates. In some embodiments, the gDNA are tagged with a first ID-tag and a second ID-tag as described above. In such embodiments, the double tagged gDNA fragments can be pooled and sequenced together using a massive parallel sequencing technique.

Depending on the specific massive parallel sequencing technology applied, the gDNA fragments may be amplified prior to sequencing. The amplification is typically performed in a clonal or spatially separated manner, so that in principle all copies of a given gDNA fragment is spatially separated from copies of other gDNA fragments. This can for example be achieved by emulsion PCR, droplet PCR, by gridded rolling circle amplication or by bridge amplification. In particular, the amplification may be performed as described herein below in the section “Amplification”.

The templates are then sequenced. Different technologies are also available for the sequencing itself, including but not limited to pyrosequencing, sequencing by reversible terminator chemistry, sequencing-by-ligation, sequencing using phospholinked fluorescent nucleotides and/or real-time sequencing.

In one embodiment of the invention the sequencing is done using reversible terminator chemistry. Such methods in general comprise synthesis of a complementary strand using single stranded gDNA fragments as template. The dNTPS employed are attached to different labels, e.g. fluorescent labels, wherein one label is used for each kind to dNTP. The labels are attached in a manner so they act as “blocking groups” and attachment should be reversible. Thus, only one dNTP is added at a time, and the kind of dNTP can be determined using the label. The label is then removed and the next dNTP is added.

Useful massive parallel sequencing platforms are commercially available and include, but are not limited to Roche 454, GS FLX Titanium, Illumina dye sequencing, Life technologies Ion proton, Complete Genomics, Helicos Biosciences Heliscope or Pacific Biosciences SMRT.

In preferred embodiments, sequencing is performed using Illumina dye sequencing, such as described in Mardis (2017).

Interestingly, the invention discloses that it is sufficient to sequence only a small random subset of the gDNA fragments. Thus, it is sufficient to sequence less than 100,000, such as less than 50,000, such as less than 20,000 gDNA fragments per sample. As noted above, typically in the range of 100 to 100,000, such as in the range of 200 to 100,000, for example in the range of 200 to 50,000, for example in the range of 1000 to 50,000, such as in the range of 5000 to 20,000 gDNA fragments or parts thereof or copies thereof as described above are sequenced. Despite the resulting genomic coverage being extremely scarce, the invention shows that the data provides an accurate sub-sample of the epigenome of each sample.

Thus, the methods in general involves a step of sequencing, wherein the number of unique tagged gDNA fragments sequenced for each sample are so few that they collectively cover no more than 10% of bases of a reference genome. The reference genome is selected so that it is from the same species as the species from which the sample is derived. Useful reference genomes are e.g. human genome hg38 or mouse mm10.

### Unique qDNA Fragments

Even though only a fraction of the gDNA fragments are sequenced, the method still allows accurate quantification of the global level of each chromatin modification of each sample.

The methods comprise a step of determining the number of unique tagged gDNA fragments from each sample and chromatin modification sub-pool. Assignment of a gDNA fragment to a specific sample may be done using the barcode sequence of the ID-tag. Assignment of the gDNA fragment to a specific chromatin modification pool may either be done by separate handling of each chromatin modification pool or by using the second barcode sequence. For each combination of sample and chromatin modification, the number of unique tagged DNA fragments are counted. This number is also referred to as “count”.

Similarly, a reference count is determined by determining the number of unique gDNA fragments of the input sub-pool for each sample.

Different criteria may be used for determining, which gDNA fragments may be considered “unique”.

In a preferred embodiment, a unique tagged gDNA fragment is a fragment identified by a unique UMI sequence and a unique mapping position in a reference genome based on the gDNA fragment sequence.

In another embodiment, it is possible to count the number of unique tagged gDNA fragments using only the unique UMI sequence. In such embodiments the gDNA sequence is not mapped to a reference genome and instead unique tagged gDNA fragments are identified solely by a unique UMI sequence. In such embodiments, the gDNA sequence itself need not be sequenced. It is sufficient to sequence the tags. Notably, in this embodiment, knowledge of the genomic sequence of the organism studied is not needed to determine the relative global levels across a plurality of samples.

In yet another embodiment, a unique tagged gDNA fragment is a fragment identified solely by a unique mapping position in the genome. In such embodiments, the use of a UMI sequence is not required.

Thus, the methods may include a step of mapping, although this is an optional step. The mapping involves mapping of the gDNA sequences to a reference genome. The reference genome is selected so that it is from the same species as the species from which the sample is derived. Useful reference genomes are e.g. human genome hg38 or mouse mm10. gDNA fragments mapping to different positions in the reference genome are considered unique. The mapping typically includes counting the number of mapped unique tagged gDNA fragments for each sample and each antibody reaction.

### Quantification of Levels of Chromatin Modification

The global levels are in general calculated as the ratio of the count of unique gDNA fragments from each sample for each immunoprecipitation compared to the count of unique tagged gDNA fragments from each sample from the input sub-pool. A higher ratio indicates a higher global level of the chromatin modification specifically recognised by the antibody used for the immunoprecipitation.

The methods of the invention are in general quantitative. Thus, the methods preferably provide a specific fold difference of any given chromatin modification compared to a reference. The specific fold difference may in particular be calculated as described below.

Typically, the ratio is calculated by determining the total count of unique sequences from each chromatin modification sub-pool. The frequency (often provided as the percentage) of unique sequences containing each of the first barcode sequences within a given chromatin modification sub-pool is then determined. Said frequency (percentage) may then be compared within each chromatin modification sub-pool to determine the fold difference. This can in particular be done if all barcode sequences are contained in the input pool at exactly or approximately the same frequency. By way of example, if 4% of the total count of unique sequences found in chromatin modification sub-pool 1 carry the first barcode of sample 1, and 2% of the total count of unique sequences found in chromatin modification sub-pool 1 carry the first barcode of sample 2, then sample 1 has twice the level of chromatin modification 1 compared to sample 2.

Frequently, individual barcodes sequences are not contained at exactly or approximately the same frequencies in the input pool. Thus, it is frequently preferred, that the methods comprise a step of calculating the input-normalized unique read count (INRC). The INRC is the frequency of unique sequences containing each first barcode sequence within a chromatin modification sub-pool normalised against frequency of unique sequences containing the same first barcode sequence in the input sub-pool. Thus, INRC can be determined by dividing the frequency of a first barcode sequence in a chromatin modification sub-pool with the frequency of the same first barcode sequence in the input sub-pool. By way of example, if 4% of the total count of unique sequences found in chromatin modification sub-pool 1 carry the first barcode of sample 1, and 2% of the total count of unique sequences found in the input sub-pool carry the first barcode of sample 1, then the INRC of sample 1 in respect of chromatin modification 1 is 2.

The fold-difference between samples may then be determined by comparing the INRC in respect of a given chromatin modification of one sample with another sample.

Thus, the ratio of a chromatin modification found in sample X compared to sample Y may be calculated using the following formula:

$$\frac{\begin{matrix}( Frequency of barcode X in chromatin modification subpool ) / \\ ( Frequency of barcode X in input subpool )\end{matrix}}{\begin{matrix}( Frequency of barcode Y in chromatin modification subpool ) / \\ ( Frequency of barcode Y in input subpool )\end{matrix}}$$

wherein the gDNA fragments of sample X are tagged with an ID-tag comprising first barcode X, and the gDNA fragments of sample Y are tagged with an ID-tag comprising first barcode Y.

The INRC and ratios of INRCs are a unit-less value which allows to compare global levels quantitatively across a plurality of samples.

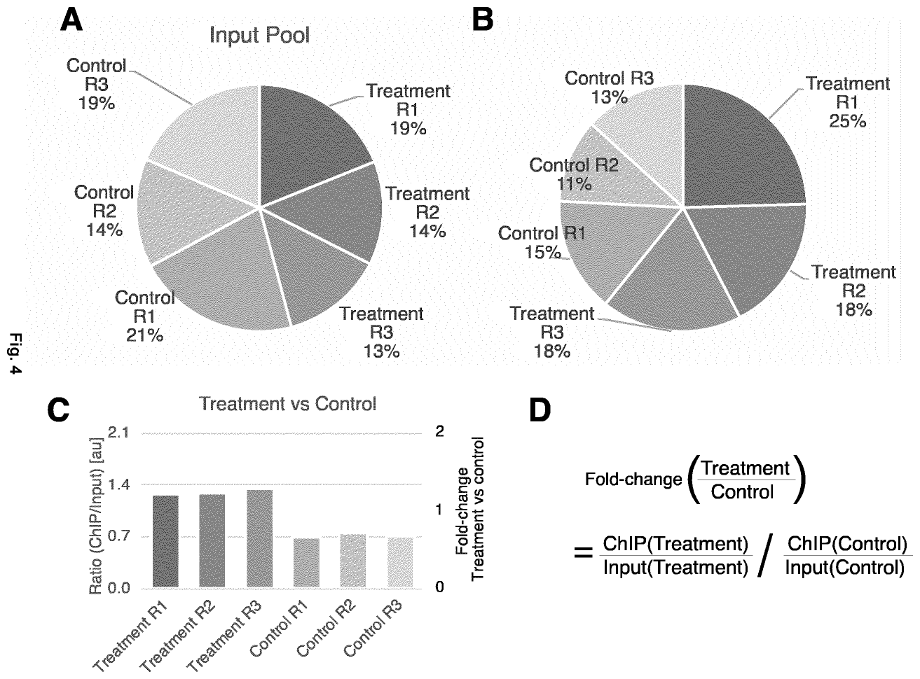

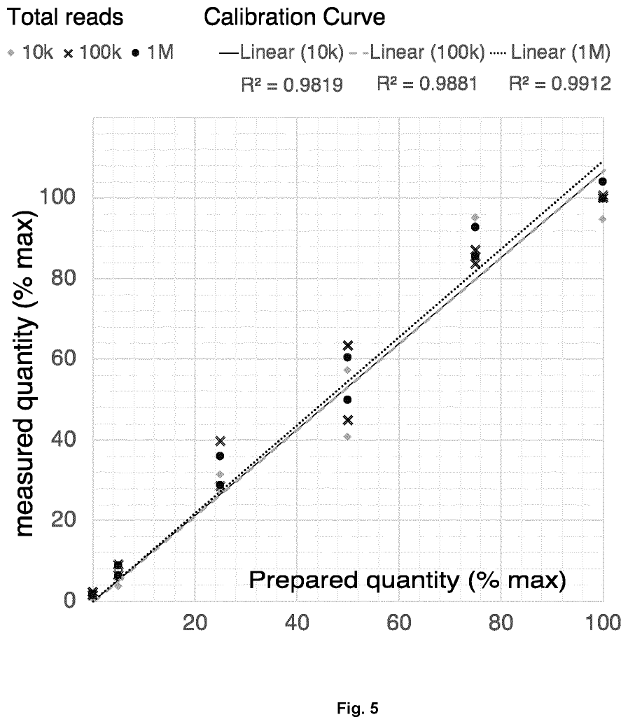

A standard curve using extremely scarce sequence data generated as described by Kumar and Elsässer, 2019, except that only a small number of randomly selected sequences were used (see Example 1, FIG. 5), demonstrates that the INRCs are in general linearly correlated or proportional to the true amount of the epitope in each sample. Thus the quantitative difference between two samples can be accurately derived as the ratio of INRCs calculated for the two samples. An example of such calculation is given in Example 2 and FIG. 4. Furthermore, FIG. 4 also describes an example of calculating the INRC as well as levels of chromatin modifications. The skilled person will be able to extrapolate from the example shown in FIG. 4, where only one treatment is shown to a setting where multiple samples are analysed.

The methods described herein yield all the quantities of a given chromatin modifications for all samples within a given pool relative to each other. In order to determine the absolute quantity of a given chromatin modification, a reference sample with a known absolute quantity of said chromatin modification may be included in the methods. If the absolute quantity of a given chromatin modification is known in a reference sample, the absolute quantity of a given chromatin modification can be accurately calculated for all the other samples in the same pool using the methods disclosed herein. A reference sample with known absolute quantity is however not required to accurately determine the levels of a chromatin modification in a plurality of samples relative to each other. A reference sample with known absolute quantity of a chromatin modification may for example be composed of synthetically produced chromatin fragments in which each gDNA is associated with one but not zero or two instances of the chromatin modification.

The present invention asserts that the quantification procedure described above can be carried out to confidently and accurately quantify the level of a chromatin modification using only a very small amount of sequencing reads, such as a number of reads between 100 to 100 000. Example 6 demonstrates that the difference in a chromatin modification between two conditions, each present in triplicate samples, can be confidently (i.e. statistically significant) and accurately determined using as little as in the range of 200 to 1000 unique reads per sample.

The methods are in particularly useful for determining the global level of chromatin modification(s) across the genome. When the methods are for determining the global level of of chromatin modification(s) across the genome, the gDNA fragments for sequencing are in principle completely randomly selected from all gDNA fragments of a sample. An example of methods for calculating global levels of chromatin modifications is shown in FIG. 4. Examples of useful tagging, amplification and sequencing steps for methods for determining global levels of chromatin modifications are provided in FIGS. 7 and 9.

However, in some embodiments, the methods are used for determining local levels of chromatin modifications. In such embodiments, only gDNA fragments from one or more loci of interest are selected for sequencing.

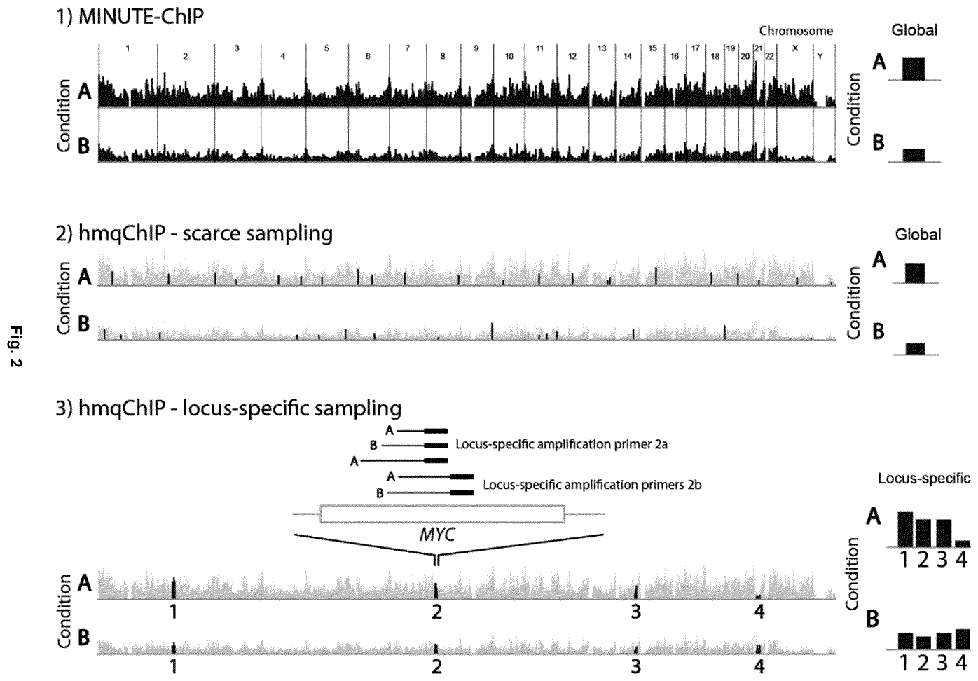

In one embodiment, the selection of gDNA fragments for sequencing is not carried out through random selection from all gDNA fragments, but via selection of chromatin fragments based on the presence of a DNA sequence complementary to a primer sequence used for selection. One or many primer sequences may be used to select one genomic locus or many loci. The methods then yield all the quantities of the chromatin modifications for all samples within a given pool relative to each other, at all the selected genomic loci. In particular, determination of the local level of chromatin modification(s) may involve a step of amplification of gDNA fragments, wherein only gDNA fragments from the loci of interest are amplified. Examples of useful tagging, amplification and sequencing steps for methods for determining global levels of chromatin modifications are provided in FIGS. 2, 8 and 10.

### Items

The invention may further be defined by any of the following items:
1. A method of assessing the levels of a plurality of chromatin modifications in parallel in a plurality of samples, said method comprising the steps of
   a. providing a plurality of test samples comprising chromatin, wherein said samples are physically separated from each other
   b. fragmenting chromatin of each sample into chromatin fragments, wherein each chromatin fragment comprises a double-stranded genomic DNA (gDNA) fragment and optionally associated proteins,
   c. tagging at least a fraction of the gDNA fragments within each sample with an ID-tag, wherein said ID-tag is an oligonucleotide which comprises a barcode sequence and optionally additional sequences, wherein gDNA fragments within one sample is tagged with a ID-tag comprising the same barcode sequence, and wherein different barcode sequences are used for each sample,
   d. combining said tagged chromatin fragments generating a pool of tagged chromatin fragments,
   e. providing a plurality of different antibodies, each specifically binding a chromatin modification,
   f. incubating each antibody with said pool of tagged chromatin fragments or a random sub-pool thereof,
   g. obtaining chromatin fragments binding each antibody, thereby obtaining a sub-pool comprising tagged gDNA fragments from chromatin fragments comprising the chromatin modification recognised by said antibody referred to as a “chromatin modification sub-pool”,
   h. optionally amplifying at least a fraction of said tagged gDNA fragments in said chromatin modification sub-pool, thereby obtaining copies of gDNA fragments, wherein said gDNA fragments and said copies thereof collectively are referred to as “gDNA fragments”;
   i. Randomly selecting in the range of 100 to 100,000 tagged gDNA fragments per sample provided in step a. from the chromatin modification sub-pools;
   j. Sequencing at least part of each of said selected tagged gDNA fragments, and determining the number of unique tagged gDNA fragments comprising each barcode sequence from each chromatin modification sub-pool,
   k. calculating the frequency of gDNA fragment comprising each barcode sequence within each chromatin modification sub-pool,
      wherein a higher frequency of gDNA fragments comprising a barcode sequence indicates a higher level of said chromatin modification in the sample tagged with ID-tags comprising said barcode sequence.
2. A method of assessing the levels of a plurality of chromatin modifications in parallel in a plurality of samples, said method comprising the steps of
   a. providing a plurality of test samples comprising chromatin from a cell population comprising a plurality of cells, wherein said samples are physically separated from each other,
   b. fragmenting chromatin of each sample into chromatin fragments, wherein each chromatin fragment comprises a double-stranded genomic DNA (gDNA) fragment and optionally associated proteins,
   c. tagging at least a fraction of the gDNA fragments within each sample with an ID-tag, wherein said ID-tag is an oligonucleotide which comprises a barcode sequence and optionally a unique molecular identifier (UMI) sequence, wherein each ID-tag comprises a different UMI sequence and optionally additional sequences, wherein gDNA fragments within one sample is tagged with a ID-tag comprising the same barcode sequence, and wherein different barcode sequences are used for each sample,
   d. combining said tagged chromatin fragments generating a pool of tagged chromatin fragments,
   e. providing a plurality of different antibodies, each specifically binding a chromatin modification,
   f. incubating each antibody with said pool of tagged chromatin fragments or a random sub-pool thereof,
   g. obtaining chromatin fragments binding each antibody, thereby obtaining a sub-pool comprising tagged gDNA fragments from chromatin fragments comprising the chromatin modification recognised by said antibody referred to as a “chromatin modification sub-pool”,
   h. optionally amplifying at least a fraction of said tagged gDNA fragments in said chromatin modification sub-pool, thereby obtaining copies of gDNA fragments, wherein said gDNA fragments and said copies thereof collectively are referred to as “gDNA fragments”;
   i. Randomly selecting in the range of n times 100 to 100,000 tagged gDNA fragments, from each chromatin modification sub-pool, wherein n is the number of samples provided in step a.; or
      pooling tagged gDNA fragments from all chromatin modification sub-pools into a combined pool and randomly selecting in the range of n times m times 100 to 100,000 tagged gDNA fragments from said combined pool, wherein n is the number of samples provided in step a. and m is the number of chromatin modification sub-pools;
   j. Sequencing at least part of each of said selected tagged gDNA fragments, and determining the number of unique tagged gDNA fragments comprising each barcode sequence from each chromatin modification sub-pool, wherein
      i. at least the first barcode sequence and
      ii. the UMI sequence and/or a part of the gDNA sequence is sequenced, and wherein a unique tagged gDNA fragments comprises either a unique UMI and/or a unique gDNA sequence,
   k. calculating the frequency of gDNA fragment comprising each barcode sequence within each chromatin modification sub-pool,
      wherein a higher frequency of gDNA fragments comprising a barcode sequence indicates a higher level of said chromatin modification in the sample tagged with ID-tags comprising said barcode sequence.
3. A method of assessing the local levels of a plurality of chromatin modifications in one or more loci of interest in parallel in a plurality of samples, said method comprising the steps of
   a. providing a plurality of test samples comprising chromatin from a cell population comprising a plurality of cells, wherein said samples are physically separated from each other
   b. fragmenting chromatin of each sample into chromatin fragments, wherein each chromatin fragment comprises a double-stranded genomic DNA (gDNA) fragment and optionally associated proteins,
   c. tagging at least a fraction of the gDNA fragments within each sample with an ID-tag, wherein said ID-tag is an oligonucleotide which comprises a barcode sequence and optionally additional sequences, wherein gDNA fragments within one sample is tagged with a ID-tag comprising the same barcode sequence, and wherein different barcode sequences are used for each sample,
   d. combining said tagged chromatin fragments generating a pool of tagged chromatin fragments,
   e. providing a plurality of different antibodies, each specifically binding a chromatin modification,
   f. incubating each antibody with said pool of tagged chromatin fragments or a random sub-pool thereof,
   g. obtaining chromatin fragments binding each antibody, thereby obtaining a sub-pool comprising tagged gDNA fragments from chromatin fragments comprising the chromatin modification recognised by said antibody referred to as a “chromatin modification sub-pool”,
   h. amplifying at least a fraction of said tagged gDNA fragments in said chromatin modification sub-pool using at least one primer specific for each locus of interest for said amplification, thereby obtaining copies of gDNA fragments, wherein said gDNA fragments and said copies thereof collectively are referred to as “gDNA fragments”;
   i. Randomly selecting in the range of 100 to 100,000 tagged gDNA fragments from the chromatin modification sub-pools, wherein n is the number of samples provided in step a. or
      pooling tagged gDNA fragments from all chromatin modification sub-pools into a combined pool and randomly selecting in the range of n times m times 100 to 100,000 tagged gDNA fragments from said combined pool, wherein n is the number of samples provided in step a. and m is the number of chromatin modification sub-pools;
   j. Sequencing at least part of each of said selected tagged gDNA fragments, and determining the number of unique tagged gDNA fragments comprising each barcode sequence from each chromatin modification sub-pool,
   k. calculating for each locus of interest the frequency of gDNA fragment comprising each barcode sequence within each chromatin modification sub-pool,
      wherein a higher frequency of gDNA fragments comprising a barcode sequence indicates a higher level of said chromatin modification at the locus of interest in the sample tagged with ID-tags comprising said barcode sequence.
4. The method according to item 3, wherein step i) comprises selecting n times p times 10 to 100,000 tagged gDNA fragments, such as n times p times 100 to 100,000, for example at the most n times p times 50,000, such as at the most n times p times 20,000, for example in the range of n times p times 10 to 50,000, such as in the range of n times p times 100 to 20,000 tagged gDNA fragments, wherein p is the number of loci of interest.
5. The method according to item 3, wherein step i) comprises selecting n times m p times 10 to 100,000 tagged gDNA fragments, such as n times m times p times 100 to 100,000, for example at the most n times m times p times 50,000, such as at the most n times m times p times 20,000, for example in the range of n times m times p times 10 to 50,000, such as in the range of n times m times p times 100 to 20,000 tagged gDNA fragments, wherein p is the number of loci of interest.
6. The method according to any one of the preceding items, wherein step d. further comprises dividing said pool into random sub-pools, wherein at least one sub-pool is an input sub-pool and the other sub-pools are test sub-pools, and wherein step f. comprises incubating each antibody with a random test sub-pool.
7. The method according to item 2, wherein said step i. further comprises randomly selecting in the range of n times 100 to 100,000 tagged gDNA fragments, wherein n is the number of samples provided in step a. from the input sub-pool, and step j. further comprises sequencing the gDNA fragments selected from the input sub-pool, and determining the number of unique gDNA fragments with each barcode sequence from the input sub-pool, and step k. further comprises determining the input normalised read count (INRC) by dividing the frequency of gDNA fragments comprising each barcode sequence within each chromatin modification sub-pool by the frequency of gDNA fragment comprising the same barcode within the input sub-pool, wherein a higher INRC of a barcode sequence indicates a higher level of said chromatin modification in the sample tagged with ID-tags comprising said barcode sequence.
8. The method according to any one of the preceding items, wherein the level of a chromatin modification in sample X compared to the level of chromatin modification in sample Y is determined by the following formula:

$$\frac{\begin{matrix}( Frequency of barcode X in chromatin modification subpool ) / \\ ( Frequency of barcode X in input subpool )\end{matrix}}{\begin{matrix}( Frequency of barcode Y in chromatin modification subpool ) / \\ ( Frequency of barcode Y in input subpool )\end{matrix}}$$

wherein the gDNA fragments of sample X are tagged with an ID-tag comprising barcode X, and the gDNA fragments of sample Y are tagged with an ID-tag comprising barcode Y.
9. The method according to any one of the preceding items, wherein said barcode sequence comprises a random sequence of in the range of 4 to 20 nucleotides, for example in the range of 6 to 16 nucleotides.
10. The method according to any one of the preceding items, wherein step j. comprises sequencing at least the barcode sequence and at least part of the gDNA sequence of the selected fragments.
11. The method according to any one of the preceding items, wherein the ID-tag comprises said barcode sequence and a unique molecular identifier (UMI) sequence, wherein each ID-tag comprises a different UMI sequence.
12. The method according to item 11, wherein the UMI comprises a random sequence of in the range of 4 to 20 nucleotides, for example in the range of 6 to 16 nucleotides.
13. The method according to any one of items 11 to 12, wherein determining the number of unique DNA fragments is done by determining the number of unique UMIs.
14. The method according to any one of the preceding items, wherein determining the number of unique tagged gDNA fragments of step j. comprises:
   i. providing the sequence of a reference genome, wherein the reference genome is from the same species as the species from which the sample is obtained
   ii. mapping the sequence of each sequenced gDNA fragment to the reference genome,
   iii. counting the number of unique gDNA fragments comprising each barcode sequence for each chromatin modification pool, wherein a unique tagged gDNA molecule is identified by a unique mapping position in the genome.
15. The method according to any one of items 11 to 14, wherein a unique tagged gDNA molecule is identified by a combination of a unique UMI and a unique mapping position in the genome.
16. The method according to any one of the preceding items, wherein the ID-tag comprises the barcode sequence, a UMI sequence and additional sequences, wherein said additional sequences comprises one or two ligation sequences, which enables ligation of the ID-tag.
17. The method according to any one of the preceding items, wherein the ID-tag comprises the barcode sequence, a UMI sequence and additional sequences, wherein said additional sequences comprises one or more amplification sequences enabling amplification of the ID-tag and/or the gDNA fragment.
18. The method according to any one of items 11 to 17, wherein step j. comprises sequencing at least the barcode sequence and the UMI sequence of each selected gDNA fragment.
19. The method according to any one of items 11 to 18, wherein step j. comprises sequencing at least the barcode sequence, the UMI sequence and the gDNA sequence of each selected gDNA fragment.
20. The method according to any one of the preceding items, wherein all first ID-tags comprises one or more common amplification sequences.
21. The methods according to any one of the preceding items, wherein the method further comprises tagging at least a fraction of the gDNA fragments with a second tag.
22. The method according to item 21, wherein said second tag is added to the opposite end of the gDNA fragment compared to the ID-tag.
23. The methods according to any one of the preceding items, wherein the method further comprises tagging at least a fraction of the gDNA fragments within each chromatin modification sub-pool with a second tag, wherein said second tag is an oligonucleotide comprising a second barcode sequence, wherein gDNA fragments within one chromatin modification sub-pool is tagged with a second tag comprising the same second barcode sequence, and wherein different second barcode sequences are used for each chromatin modification sub-pool.
24. The method according to any one of items 21 to 23, wherein gDNA fragments within the input sub-pool are tagged with the same second barcode sequence, which is different to the second barcode sequence used for the chromatin modification sub-pools.
25. The method according to any one of items 21 to 24, wherein the method further comprises a step of pooling the gDNA fragments of one or more chromatin modification sub-pool and the input sub-pool into a combined pool, and wherein step i) comprises selecting in the range of n times m times 100 to 100,000 tagged gDNA fragments from said combined pool.
26. The method according to any one of items 21 to 25, wherein the method further comprises a step generating a pool of gDNA fragments by pooling the gDNA fragments of all chromatin modification sub-pools and the input sub-pool.
27. The method according to any one of items 25 to 26, wherein step i. comprises randomly selecting in the range of 100 to 100,000 gDNA fragments per sample provided in step a. from said combined pool.
28. The method according to any one of items 25 to 26, wherein step i. comprises randomly selecting in the range of 200 to 100,000 gDNA fragments per sample provided in step a. from said combined pool.
29. The method according to any one of the preceding items, wherein step i. comprises randomly selecting in the range n time m times 200 to 100,000 gDNA fragments from said combined pool.
30. The method according to any of the preceding items, wherein step i. comprises randomly selecting at the most 50,000, such as at the most 20,000, for example in the range of 1000 to 50,000, such as in the range of 5000 to 20,000 gDNA fragments per test sample provided in step a.
31. The method according to any of the preceding items, wherein step i. comprises randomly selecting at the most n times 50,000, such as at the most n times 20,000, for example in the range of n times 1000 to 50,000, such as in the range of n times 5000 to 20,000 gDNA fragments.
32. The method according to any one of items 21 to 31, wherein step j. comprises sequencing at least the barcode sequence of the ID-tag and the second barcode sequence and the UMI sequence and/or the gDNA sequence, and wherein step j. comprises calculating the frequency of unique gDNA fragments comprising the barcode sequence of the ID-tag and each specific second barcode sequence in relation total number of unique gDNA fragments comprising said specific second barcode sequence.
33. The method according to any one of the preceding items, wherein the method comprises step h.
34. The method according to any one of the preceding items, wherein said amplification is performed by a method comprising polymerase chain reaction (PCR), linear amplification and/or reverse transcription using at least one primer capable of annealing to an amplification sequence in the ID tag.
35. The method according to any one of items 21 to 34, wherein the second tag is added during the amplification of step h.
36. The method according to any one of the preceding items, wherein the second tags comprises one or more common amplification sequence to which a primer can anneal.
37. The method according to any one of the preceding items, wherein at least one of the primers for amplification comprises an adaptor tag.
38. The method according to any one of the preceding items, wherein said amplification is performed by a method using one primer capable of annealing to the amplification sequence in the second tag.
39. The method according to any one of the preceding items, wherein said randomly selected gDNA fragments may be immobilised on one or more solid supports.
40. The method according to any one of the preceding items, wherein the ID-tag contains at the most 100 nucleotides, such as at the most 75 nucleotides, for example at the most 50 nucleotides.
41. The method according to any one of the preceding items, wherein the ID-tag consists of an oligonucleotide consisting of in the range of 6 to 100 nucleotides, such as in the range of 6 to 75 nucleotides, for example in the range of 6 to 50 nucleotides.
42. The method according to any one of the preceding items, wherein said chromatin is fragmented by incubation with one or more enzymes catalysing fragmentation of chromatin.
43. The method according to item 42, wherein said one or more enzymes are selected from the group consisting of
   a. nucleases, such as micrococcal nuclease (MNase)
   b. sequence-specific restriction enzymes
44. The method according to any one of the preceding items, wherein said chromatin is fragmented by mechanical shearing, such as by sonication.
45. The method according to any one of the preceding items, wherein tagging said gDNA fragment with said ID-tag and/or said second tag is done by ligation.
46. The method according to any one of the preceding items, wherein tagging comprises ligation of the ID-tag and/or second tag using adaptor ligation using DNA ligase, such as using T4 DNA ligase.
47. The method according to any one of the preceding items wherein tagging comprises ligation of the ID-tags and/or second tags using adaptor ligation using a transposon enzyme, such as using Tn5.
48. The method according to any one of the preceding items, wherein step a. comprises providing at least 15, such as at least 25, for example at least at least 50, such as at least 75, for example in the range of 15 to 1000, such as in the range of 15 to 500, for example in the range of 25 to 1000, such as in the range of 25 to 500 different test samples comprising chromatin are provided.
49. The method according to any one of the preceding items, wherein step a. comprises providing at least 75, preferably at least 85, for example in the range of 75 to 1000, such as in the range of 75 to 500, for example providing in the range of 85 to 1000, such as in the range of 85 to 500 different test samples comprising chromatin.
50. The method according to any one of the preceding items, wherein the test samples comprises cells, for example cells selected from the group consisting of
   a. transformed cell lines;
   b. primary cell lines, such as patient derived cell lines;
   c. cancer cell lines;
   d. iPS cells;
   e. adherent cells;
   f. suspension cells;
   g. 3D cell cultures;
   h. cells of engineered tissues and
   i. cell of organoids
51. The method according to any one of the preceding items, wherein the cell population comprises at least 100 cells, preferably at least 500 cells, even more preferably at least 1000 cells, for example in the range of 10 to 100,000 cells, such as in the range of 100 to 100,000 cells, for example in the range of 1000 to 100,000 cells.
52. The method according to any one of the preceding items, wherein each sample comprises in the range of 100 to 100000 cells, such as in the range of 1000 to 10000 cells.
53. The method according to any one of the preceding items, wherein each sample comprises cells, which have been subjected to a different treatment.
54. The method according to any one of the preceding items, wherein one or more chromatin modifications are selected from the group consisting of:
   i. a protein present within the chromatin fragment
   ii. a post-translational modifications
   iii. a modification of a nucleotide
   iv. presence of a non-natural nucleo-base in the gDNA fragment
   v. presence of a protein fragment produced through post-translational processing
   vi. a non-canonical DNA structure
55. The method according to any one of the preceding items, wherein one or more chromatin modifications is methylation of a nucleotide.
56. The method according to any one of the preceding items, wherein one or more modifications of said nucleotide leads to the presence of one or more modified nucleotides selected from the group consisting of
   a. 5-methyl-cytosine
   b. 5-hydroxy-methyl-cytosine
   c. 5-formyl-cytosine
   d. 5-carboxycytosine; and
   e. 6-methyl-adenine
57. The method according to any one of items 46 to 48, wherein one or more non-natural nucleobase are selected from the group consisting of 5-brom-2′-deoxiuridin and 5-Ethynyl-2′-deoxyuridine base.
58. The method according to any one of items 46 to 48, wherein one or more non-canonical DNA structures are selected from the group consisting of a G4 structure, a single-stranded DNA, and a RNA:DNA hybrid.
59. The method according to any one of the preceding items, wherein step e. comprises providing at least 5 different antibodies, such as at least 10 different antibodies, for example at least 15 different antibodies, such as in the range of 5 to 100 different antibodies, for example in the range of 5 to 50 different antibodies, such as in the range of 10 to 100 different antibodies, for example in the range of 10 to 50 different antibodies each specifically binding a different chromatin modification.
60. The method according to any one of the preceding items, wherein one or more antibodies specifically and selectively binds a posttranslational modification selected from the group consisting of carboxylation, methylation, hydroxymethylation, acetylation, glutamylation, citrullination, phosphorylation and glycosylation of an amino acid.
61. The method according to any one of the preceding items, wherein one or more antibodies specifically and selectively binds a posttranslational modification comprising an isomerisation, for example proline isomerization, or formation of atypical isoaspartyl.
62. The method according to any one of the preceding items, wherein at least 5 different antibodies binding different histone PTMs, such as at least 10 different antibodies binding different histone PTMs, for example at least 15 different antibodies binding different histone PTMs, such as in the range of 5 to 100 different antibodies binding different histone PTMs, for example in the range of 5 to 50 different antibodies binding different histone PTMs, such as in the range of 10 to 100 different antibodies binding different histone PTMs, for example in the range of 10 to 50 different antibodies binding different histone PTMs are provided.
63. The method according to any one of the preceding items, wherein one or more antibodies specifically and selectively binds an posttranslational modification selected from the group consisting of
   a. Methylated (mono-, di-, tri-methylated) lysine;
   b. acylated (acetylated, propionylated, butyrylated, isobutylated, succinylated, crotonylated, hydroxyisobutyrylated) lysine;
   c. ubiquitinated lysine;
   d. sumoylated lysine;
   e. neddylated lysine;
   f. phosphorylated serine;
   g. phosphorylated threonine;
   h. phosphorylated histidine;
   i. citrulline;
   j. methylated arginine (mono-, symmetric di-, assymetric dimethylation)
   k. glutarylated lysine;
64. The method according to any one of the preceding items, wherein one or more antibodies specifically and selectively binds a posttranslational on a protein selected from the group consisting of
   a. Histone H3
   b. Histone H3.1, H3.2, H3.3
   c. Histone H3.X, H3.Y
   d. Histone H4
   e. Histone H2A
   f. Histone H2A.X
   g. Histone H2A.Z
   h. Histone H2A.Z.1
   i. Histone H2A.Z.2
   j. Histone macroH2A
   k. Histone H2A.Bbd; and
   l. Histone H2B.
65. The method according to any one of the preceding items, wherein one or more antibodies specifically and selectively binds an epigenetic modification selected from the group consisting of
   a. H3K4me1;
   b. H3K4me2;
   c. H3K4me3;
   d. H3K79me3;
   e. H3K9me1;
   f. H3K9me2;
   g. H3K9me3;
   h. H3K27me1;
   i. H3K27me2;
   j. H3K27me3;
   k. H4K20me1;
   l. H4K20me2 and
   m. H4K20me3.
66. The method according to any one of the preceding items, wherein sequencing is performed by massive parallel sequencing.
67. The method according to any one of the preceding items, wherein sequencing is performed by Illumina sequencing.
68. The method according to any one of the preceding items, wherein the method comprises randomly selecting and sequencing of in the range of 100 to 100,000, for example at the most 50,000, such as at the most 20,000, for example in the range of 1000 to 50,000, such as in the range of 5000 to 20,000 tagged gDNA fragments per test sample.
69. The method according to any one of the preceding items, wherein the method comprises randomly selecting and sequencing of in the range of 200 to 100,000, for example at the most 50,000, such as at the most 20,000, for example in the range of 200 to 50,000, for example in the range of 1000 to 50,000, such as in the range of 5000 to 20,000 tagged gDNA fragments per test sample.
70. The method according to any one of the preceding items, wherein the method comprises randomly selecting and sequencing of in the range of n times 100 to 100,000, for example at the most n times 50,000, such as at the most n times 20,000, for example in the range of n times 1000 to 50,000, such as in the range of n times 5000 to 20,000 tagged gDNA fragments.
71. The method according to any one of the preceding items, wherein the method comprises randomly selecting and sequencing of in the range of n times 200 to 100,000, for example at the most n times 50,000, such as at the most n times 20,000, for example in the range of n times 200 to 50,000, for example in the range of n times 1000 to 50,000, such as in the range of n times 5000 to 20,000 tagged gDNA fragments per test sample.
72. The method according to any one of the preceding items, wherein the method comprises randomly selecting and sequencing of in the range of n time m times 100 to 100,000, for example at the most n time m times 50,000, such as at the most n time m times 20,000, for example in the range of n time m times 1000 to 50,000, such as in the range of n time m times 5000 to 20,000 tagged gDNA fragments, wherein n is the number of test samples provided in step a., and m is the number of chromatin modification sub-pools.
73. The method according to any one of the preceding items, wherein the method comprises randomly selecting and sequencing of in the range of n time m times 200 to 100,000, for example in the range of n time m times 200 to 50,000, for example in the range of n time m times 1000 to 50,000, such as in the range of n time m times 5000 to 20,000 tagged gDNA fragments, wherein n is the number of test samples provided in step a., and m is the number of chromatin modification sub-pools.
74. The method according to any one of items 3 to 61, wherein the INRCs are linearly related to the true amount of the epitope in each sample, and the quantitative difference between two samples corresponds to the ratio of the INRCs calculated for the two samples.
75. The method according to any one of the preceding items, wherein the method is for determining global levels of a plurality of chromatin modifications, and wherein gDNA fragments are randomly selected from all gDNA fragments of each sample.
76. The method according to any one of the preceding items, wherein the method is for determining local levels of a plurality of chromatin modifications, and wherein gDNA fragments are randomly selected from gDNA fragments from one or more genomic loci of interest.
77. The method according to any one of the preceding items, wherein the method is a method for quantitatively assessing the levels of a plurality of chromatin modifications.
78. The method according to any one of the preceding items, wherein said quantitative assessment provides the fold difference of each chromatin modification compared to a reference.
79. A method of determining the influence of test compounds on the levels of a plurality of chromatin modifications, said method comprising the steps of
   a. Providing one or more test compounds;
   b. Cultivating cells in the presence of said test compounds, wherein cells cultivated in the presence of different test compounds are physically separated from each other;
   c. Performing the method according to any one of items 1 to 78, wherein each test sample comprises cells incubated with a different test compound.
80. A method of determining the influence of test compounds on the level of a plurality of chromatin modifications, said method comprising the steps of
   a. Providing one or more test compounds;
   b. Cultivating a plurality of cells in the presence of said test compounds or combinations thereof, wherein cells cultivated in the presence of different test compounds or combinations thereof are physically separated from each other, and wherein cells cultivated in the presence of a given test compound or combination thereof is a cell population;
   c. Performing the method according to any one of items 1 to 78, wherein each test samples comprises chromatin from different cell populations.
81. The method according to any one of items 79 to 80, wherein the method further comprises performing the method according to any one of items 1 to 78 with a reference sample comprising cells, which have not been incubated with a test compound.
82. The method according to item 81, wherein the influence of test compound(s) on the level of a plurality of chromatin modifications is determined by comparing the frequency of said chromatin modification in a cell population cultivated in the presence of said test compound(s) compared to frequency in the reference sample.

### EXAMPLES

The invention may further be illustrated by the following examples, which however should not be construed as limiting for the invention.

### Example 1

The example presented below is based on a dataset prepared according to Kumar and Elsässer, Cell Reports, 2019, except that only a small number (N) of randomly selected sequences were used.

First, calibration curves were prepared by mixing at known ratios a cell source with maximal levels of H3K27me3 with a cell source depleted of H3K27me3. The respective sources were generated by treating mouse embryonic stem cells (mESC) either with EZH2 inhibitor EPZ-6438, to reduce H3K27me3 below detectable levels, or with an inhibitor to demethylases JMJD3/UTX, GSK-J4, to increase H3K27me3 above physiologic levels. Samples with pre-set very high or very low quantities of histone H3K27me3 were prepared by mixing these cell sources at 7 known ratios (100% Low, 5% High/95% Low, 25%High/75% Low, 50% High/50% Low, 75% High, 25% Low, 95% High, 5% Low, 100% Hich). Two replicates were prepared for each ratio and barcoded independently using 14 different first barcodes. The lysis, chromatin fragmentation and barcoding, chromatin immunoprecipitation and library preparation was carried out according to the MINUTE-ChIP method described in Kumar and Elsässer 2019. In Kumar et. al., ˜600 Mio unique sequences (read pairs) were acquired on an Illumina platform, corresponding to an average of ˜10 Mio sequences per datapoint (i.e. per sample and pool). According to the theory described above, the number of sequences in each H3K27me3 ChIP sample shall be proportional to the pre-set amount of H3K27me3 in the sample: For the sample with lowest H3K27me3, 251,861 sequences were acquired, for the sample with highest H3K27me3, 48,715,703 sequences were acquired. Hence, in Kumar et. al., the level of H3K27me3 in each sample was accurately determined with an average of ˜10 Mio sequences per datapoint. To demonstrate that the same quantification can be achieved with a randomly selected small number of reads, the effect of using a very small number of sequences was tested: In the first analysis, 250,000 sequences were randomly selected from each library (Input, H3K27me3 ChIP), corresponding to an average of 17857 raw sequences. This provided an average of 15,909 (ranging from 270 to 68,737) unique, mapped sequences per datapoint (i.e. per sample per library). In another analysis, 25,000 sequences were randomly selected, corresponding to an average of 1786 raw sequences. This provided an average of 1,633 (ranging from 26 to 6,938) unique, mapped sequences per datapoint (i.e. per sample per library). In a third analysis, 2,500 sequences were randomly selected, corresponding to an average of 179 raw sequences. This provided 166 (ranging from 2 to 716) unique, mapped sequences per datapoint (i.e. per sample per library). The resulting global quantifications based on INRC are shown in FIG. 5. As shown in the figure the measurement is linearly correlated with and proportional to the pre-set quantity. The R² value, an indicator of how closely the quantities data predicts the true quantities, remains extremely good (>0.98) even when using only 5,000 reads in total (2,500 from the H3K27me3 ChIP sub-pool, 2,500 from the Input sub-pool) corresponding to an average 357 sequences per sample) to produce the standard curve. The calibration curve quantification demonstrates that the INRC is proportional to the global abundance across a large dynamic range.

In the next experiment, cells grown under two conditions, each with triplicate samples were compared against each other, determining the relative levels of the histone epitope, H3K27me3. Thus a total of 6 samples were studied, each being barcoded with a different first barcode (6 barcodes in total).

The comparison studied here refers to two conditions, one ‘untreated’ condition representing mouse embryonic stem cells grown in standard medium and a “2i” inhibitor treatment representing mouse embryonic stem cells grown in standard medium with two specific inhibitors, namely MEK inhibitor PD0325901 and GSK3 inhibitor CHIR99021 (“2i”). Quantification using a total of ˜1000 Mio sequences demonstrated that cells in “2i” treatment lead to a statistically significant (t Test, p=0.0017) 2.3-fold increase in H3K27me3 (Kumar and Elsässer, Cell Reports, 2019).

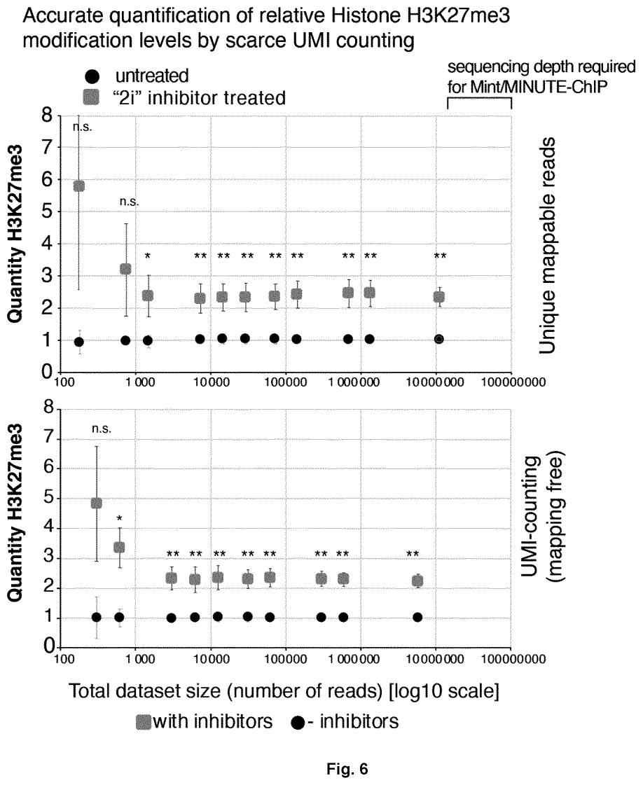

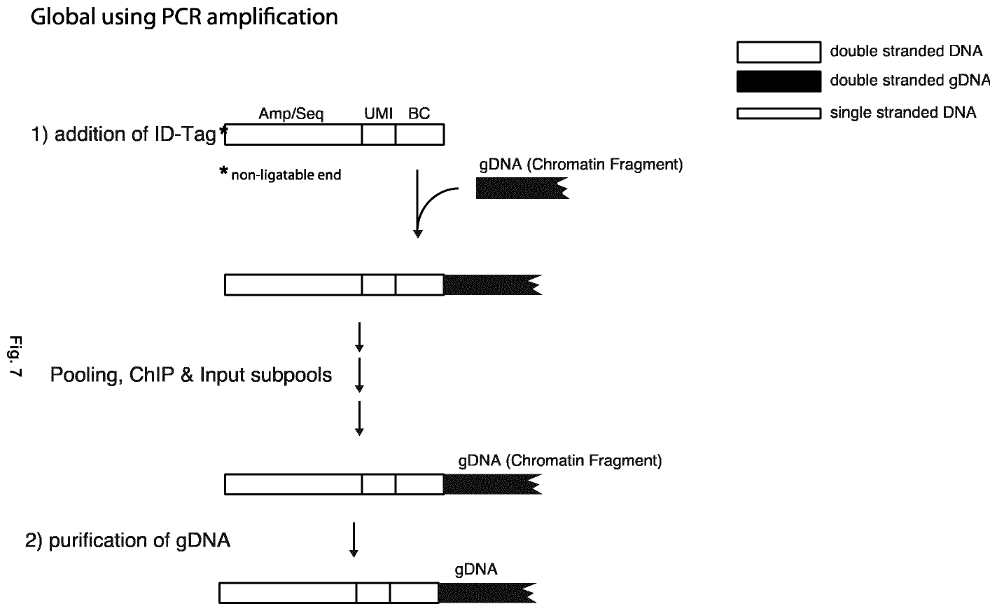

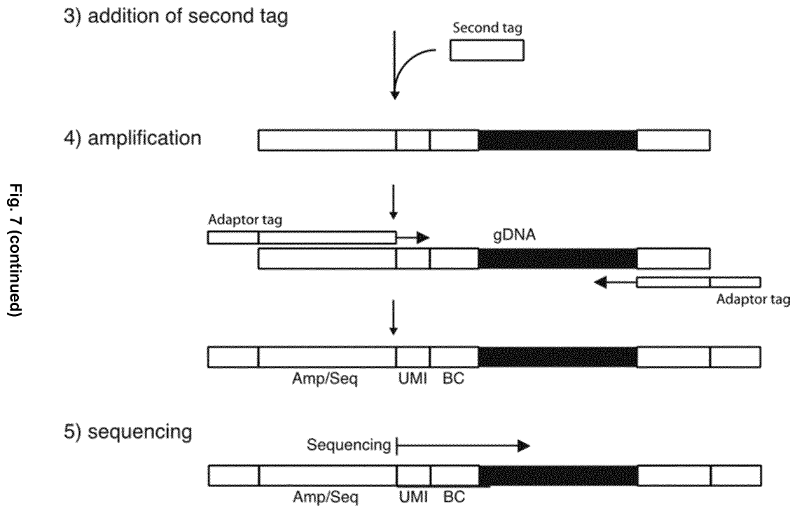

Different numbers of sequences were randomly selected from the H3K27me3 sub-pool library and the input sub-pool library. Approximately twice as many sequences were selected from the H3K27me3 sub-pool as compared to the input sub-pool, and a total number of sequences spanning the range of 200 to 10,000,000 randomly selected sequences were analysed, and the results are shown in FIG. 6.

Analysing as little as 3043 randomly selected sequences in total (2017 from the H3K27me3 sub-pool, 1026 from the Input sub-pool) demonstrates that both the calculated fold-change as well as the standard deviations of the measurement and significance of the pairwise comparison are robust against a dramatic reduction of underlying read information. Within the 3043 selected sequences, individual samples were represented with minimally 101 and maximally 606 unique sequences per pool) Hence, sampling at a low depth, between 1000 and 10 000 reads in total across the immunoprecipitation (histone modification subpool) and Input sub-pool (corresponding to 100 to 1500 reads per sample per sub-pool) still allowed a statistically significant determination of the 2.3-fold change (see FIG. 6).

Further, quantitation based on mapped unique reads counts) and UMI information only was compared, demonstrating that UMI-counting yielded the same result with the same statistical significance while using more of the available sequencing information (since all reads contain UMI information but not all can be mapped to the reference genome). Sampling 1484 randomly selected sequences (754 for H3K27me3 sub-pool, 730 for the Input sub-pool) still allowed a statistically significant determination of the 2.3-fold change (see FIG. 6). Within the 1484 selected sequences, individual samples were represented with minimally 30 and maximally 246 unique sequences per pool.

### Example 2

### Quantification of Global Levels

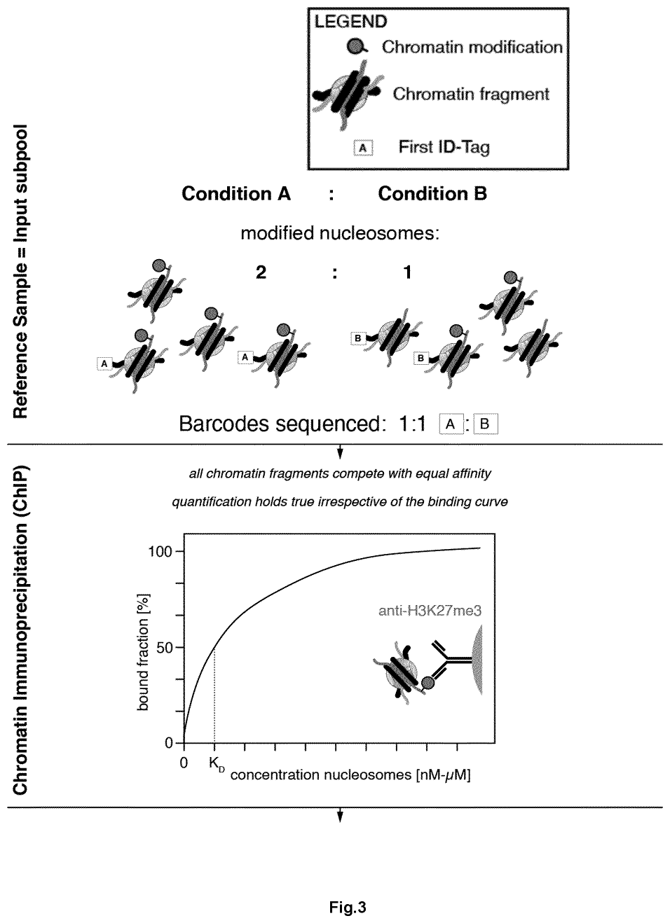

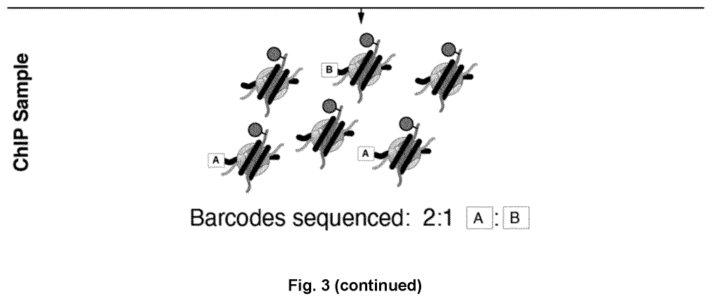

The present example aim to provide insight into the step of quantifying the global levels. A multiplexed, pooled ChIP essentially represents a competitive binding experiment. The present example is given with two conditions. The modification H3K27me3 is twice as abundant in condition A as compared to B. After fragmentation of chromatin, twice as many nucleosome molecules carry a H3K27me3. The nucleosomal DNA is barcoded according to source (“A” or “B”) before pooling. When the input pool is sequenced, barcodes “A” and “B” are observed with the same frequency. For the ChIP reaction, the pool is added to specific antibodies immobilized on beads, providing a limited number of binding sites for H3K27me3. Since H3K27me3 is more abundant in condition A, nucleosomes with the “A” barcode are more likely to be captured by the antibody. The bound nucleosomes are sequenced. Barcode “A” will be observed twice as often as barcode “B”. The barcode distribution is always proportional to the relative abundance of the epitope in the input samples because of the competition for binding sites (FIG. 3).

To quantify the level present in each sample of the epitope assayed in a given IP reaction, unique combinations of the sample specific tag and UMI are counted and summed up for each sample. These total unique read counts determined for each sample specific tag are then related to the total unique reads counts of the same sample specific tag in the input, to form the input normalized read count (INRC). According to the above example calculation the INCR is proportional to the amount of the epitope in each sample.

In this version of method, unique barcode molecules are determined, assessing only the UMI information. In an alternative version of the method, the genomic sequence content of the read (the sequence corresponding to the chromatin fragment that was ligated to the specific adaptor molecule) is mapped to the genome, and unique molecules are determine by a unique genomic sequence in combination with a unique UMI.

### Example 3

### hmqChIP Experiment for Quantifying Drug Effect on Three Histone Modifications

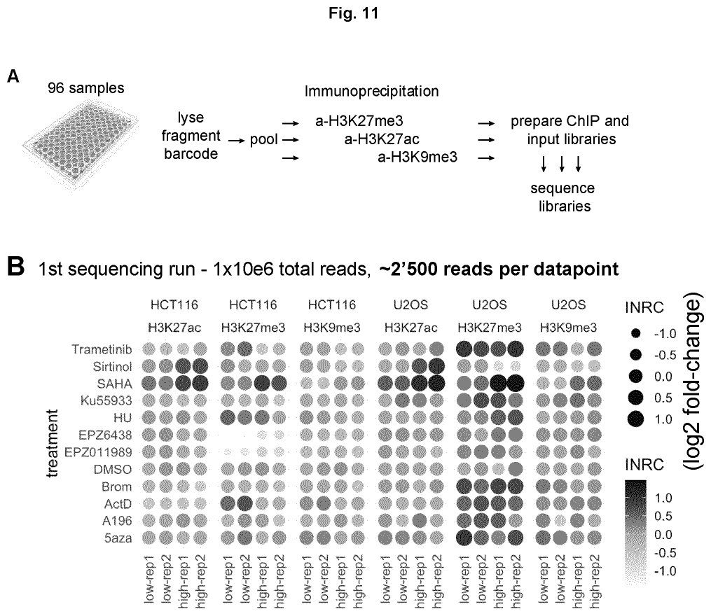

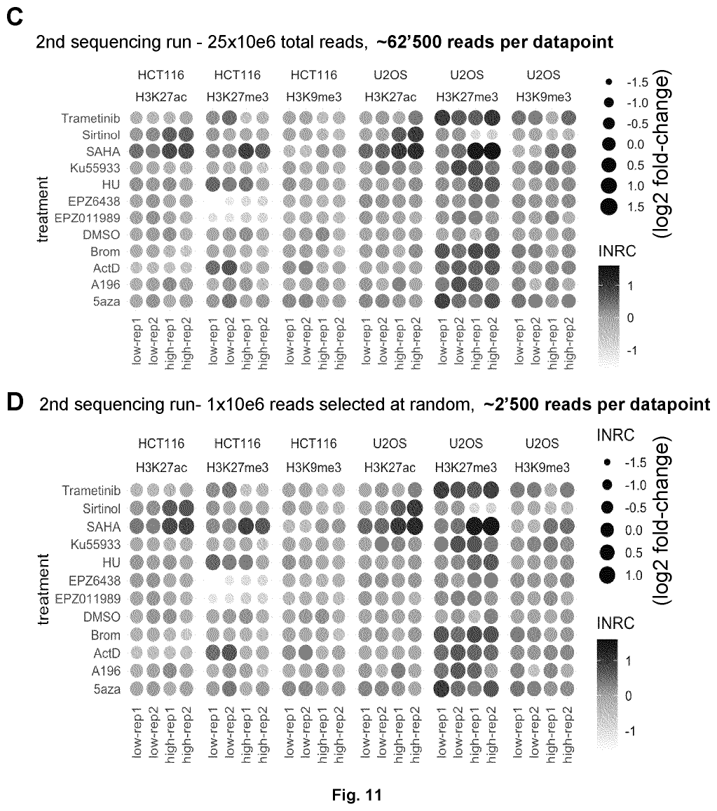

A highly multiplexed experiment profiling global levels of three histone modifications in 96 samples: Two different cancer cell lines (human colon cancer HCT116, human osteosarcoma U2OS) were treated with 11 different drugs at 2 concentrations each (low, high) or DMSO as a control. Two replicates each (rep1, rep2) were performed of each treatment, totaling 96 samples. An overview of the experiment is shown in FIG. 11A. For U2OS, 9000 cells were seeded per 96-well, For HCT116, 6000 cells were seeded. After 24 h, drugs were added to each well, using the concentration specified in the following table:

| | Stock | | Final conc. | |
|---|---|---|---|---|
| Drug | conc. | | (low/high) | Description |
| DMSO | — | | — | Control |
| SAHA | 100 | uM | 0.5 uM/5 uM | HDACi. Pan-class I and class |
| | | | | II HDAC inhibitor |
| Sirtinol | 10 | mM | 0.5 uM/5 uM | SIRT1 & 2 inhibitor |
| Hydroxyurea (HU) | 2M | | 0.5 uM/5 uM | DNA DAMAGE, yH2AX |
| EPZ011989 | 5 | mM | 0.5 uM/5 uM | EZH2 inhibitor |
| 5-Aza-2′-deoxycytidine/ | 25 | mM | 0.5 uM/5 uM | HDACi |
| Decitabine | | | | |
| Ku55933 | 10 | mM | 0.5 uM/5 uM | DNA DAMAGE, yH2AX |
| | | | | (ATMi) |
| Actinomycin D | 1 | mM | 1 nM/5 nM | DNA DAMAGE, yH2AX |
| | | | | (RNA pol I, and RNA pol II |
| | | | | inhibitor) |
| Trametinib | 20 | mM | 0.5 uM/5 uM | MEK1/2 inhibitor |
| Bromosporine | 10 | mM | 0.5 uM/5 uM | Broad spectrum inhibitor |

| | | | |
|---|---|---|---|
| | | | for bromodomains |
| EPZ 6438/Tazemetostat | — | 0.5 uM/5 uM | EZH2 inhibitor |
| A-196 | — | 0.5 uM/5 uM | SUV420H1 and SUV420H2 |
| | | | (methyltransferase) inhibitor |

After further growth for 40 h, the cell number was approx. 50 000 cells per well. Cells were directly lysed in the culture plate, chromatin was fragmented and barcoded with 96 ID-tags comprising 96 different first barcodes (one barcode for each sample) in the same culture plate as follows: The growth medium was removed and the well was washed 1× with PBS. 30 uL lysis buffer (50 mM Tris-HCl, pH 8.0, 0.1% Triton X-100, 0.05% sodium deoxycholate (DOC), 5 mM CaCl2 was added per well. The plate was placed on ice for 15 min. 20 uL lysis buffer with 15 U/uL MNase was added. The plate was incubated at 37 C for 20 min. 13 uL ddH2O and 7 uL blunting master mix (10 mM ATP, 10 mM dNTP, 50 mM DTT, 15 U T4 DNA Polymerase, 25 U T4 Polynucleotide Kinase, 300 mM EGTA) were added to each well, raising the total volume to 70 uL. The plate was incubated at room temperature for 1 h. 2 uL of 1.25 uM DNA adaptor was added to each well. The adaptor sequence (ID-tag) was as described in Kumar and Elsässer, 2019 with a unique 8 nt barcode for each sample and 6 nt random UMI sequence.

18 uL of ligation reaction mix (5.4 uL 50% w/v PEG 4000, 0.45 uL 100 mM ATP, 0.5 uL T4 DNA Ligase (5 U/uL), 11.65 uL ddH2O) was added to each well, totalling 90 uL. The plate was incubated 1 h at room temperature. The reaction was stopped by adding 90 uL stop buffer (100 mM Tris-HCl pH 8.0, 300 mM NaCl, 2% Triton X-100, 100 mM EGTA, 100 mM EDTA, 0.2% sodium deoxycholate) and the wells of the 96 w plate were pooled into a single tube. The pool of 17,820 uL was aliquoted in 1.5 mL aliquots and spun at 2000 rpm for 5 min, the supernatant was recovered. Subpools of 1.5 mL were incubated with 3 uL anti-H3K27me3 (Millipore 07-449), anti-H3K27ac (Abcam ab177178), anti-H3K9me3 (Abcam ab8898) antibody immobilized on 50 uL Protein A/G beads. DNA from ChIP and Input was isolated and purified as described in Kumar and Elsässer, 2019. Libraries were prepared with 75% of the purified DNA as described in Kumar and Elsässer, 2019.

The final libraries were sequenced twice, to test the influence of sequencing two different random set of molecules on the quantification of global levels (that only scarcely covering the genome). The first time ˜1 Mio reads were sequenced in total for the four sub-pools (3 ChIP, 1 input), approximately 250 000 per sub-pool. This corresponds to sequencing ˜10,416 sequences per sample, or 2604 sequences per datapoint (i.e. per sample per library). The second time at total of 25 Mio reads were collected and either all of the reads were analyzed, or only a randomly selected subset of 1 mio reads. When all reads were analyzed, it corresponds to approx. 260,416 reads per sample. Input-normalized unique read count (INRCs) using DMSO controls as reference were calculated as described in Example 2 and plotted in a heatmap with grayscale and circle size reflecting the INRC.

The results are shown in FIG. 11. FIG. 11B shows the results of first sequencing, where ˜1 Mio reads were sequenced, whereas FIG. 11C shows the results of the second sequencing run using only 1 Mio randomly selected reads out of 25 Mio. This corresponds to analysing ˜10,416 reads per sample. FIG. 11D shows the results of the second sequencing run using 25 Mio reads.

The extremely low sequencing depth of 10,000 sequences per sample or 2500 sequences per datapoint (i.e. per sample per sub-pool) in FIGS. 11B) and C) produced the same results as the 25× deeper sequencing in FIG. 11D). Hence, extremely low sequencing depth still yield reliable global quantification. As expected, the HDAC inhibitors SAHA (Vorinostat) and Sirtinol increase H3K27ac acetylation. SAHA inhibits HDAC1-HDAC10, whereas Sirtinol inhibits SIRT1 an SIRT2. Also as expected, the EZH2 inhibitors EPZ6438 and EPZ011989 decrease H3K27me3 in HCT116 cells. U2OS cells were however unaffected by the EZH2 inhibitors, presumably pre-existing H3K27me3 is not diluted or removed quickly, in line with the relative slow growth of U2OS cells. Interestingly, despite both HDAC inhibitors increase H3K27ac, only Sirtinol at the same time decreases H3K27me3 in U2OS cells, presumably by stimulating active demethylation. By testing all conditions against all epigenetic markers, known and novel relationships are systematically discovered.

### Example 4

### hmqChIP Experiment for Locus Specific Quantification of Chromatin Modifications

From the experiment described above in Example 3, 25% of the purified DNA from H3K9me3 ChIP, H327ac ChIP and Input was amplified using PCR with the universal forward primer as described in Kumar and Elsässer, 2019, and a mix of reverse primers hybrid complementary to sequences in the CDKN1A (p21) promoter locus (+/−2.5 kb). (agacgtgtgctcttccgatctCGGTGGGAAAGAGGTAGAG (SEQ ID NO:1), agacgtgtgctcttccgatctGTGTCCCGGACCTCCAGT (SEQ ID NO:2), agacgtgtgctcttccgatctCTCGCTAGTCCTTAGGGGA (SEQ ID NO:3), agacgtgtgctcttccgatctCAGGGACACGGACTTCAT (SEQ ID NO:4), agacgtgtgctcttccgatctCATCCCGACTCTCGTCAC (SEQ ID NO:5)) The final library PCR was subsequently performed as in Kumar and Elsässer, 2019.

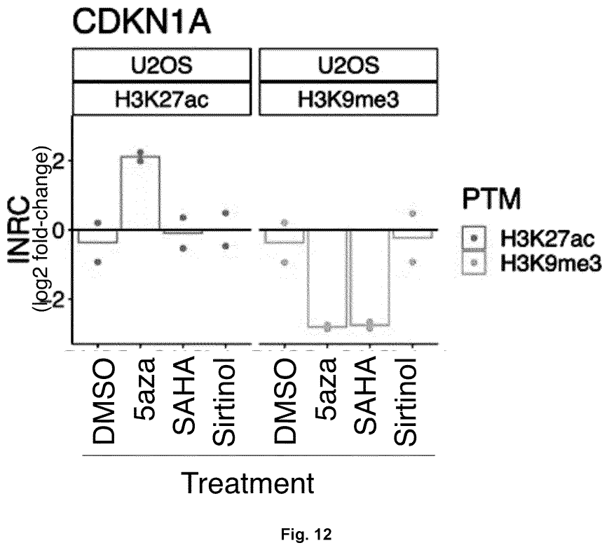

˜10 000 sequences were collected on an Illumina sequencer for each library. Reads were aligned to the human genome and sequences aligning to the CDKN1 promoter (+/−2.5 kb) were counted yielding 2 to 500 reads per sample per ChIP. The INRC was calculated for selected conditions (DMSO, 5aza, SAHA, Sirtinol) using only the sequences aligning to the CDKN1 promoter as described in Example 2, and plotted in FIG. 12, using the DMSO control as reference condition.

### REFERENCES

Kumar and Elsässer, Cell Reports, 2019, 28(12):3274-3284. doi: 10.1016/j.celrep.2019.08.046.

Mardis E. DNA sequencing technologies: 2006-2016. Nat Protoc 12, 213-218 (2017). https://doi.org/10.1038/nprot.2016.182

1. A method of assessing the levels of a plurality of chromatin modifications in parallel in a plurality of samples, said method comprising the steps of
a. providing a plurality of test samples comprising chromatin from a cell population comprising a plurality of cells, wherein said samples are physically separated from each other,
b. fragmenting chromatin of each sample into chromatin fragments, wherein each chromatin fragment comprises a double-stranded genomic DNA (gDNA) fragment and optionally associated proteins,
c. tagging at least a fraction of the gDNA fragments within each sample with an ID-tag, wherein said ID-tag is an oligonucleotide which comprises a barcode sequence and optionally a unique molecular identifier (UMI) sequence, wherein each ID-tag comprises a different UMI sequence and optionally additional sequences, wherein gDNA fragments within one sample is tagged with a ID-tag comprising the same barcode sequence, and wherein different barcode sequences are used for each sample,
d. combining said tagged chromatin fragments generating a pool of tagged chromatin fragments,
e. providing a plurality of different antibodies, each specifically binding a chromatin modification,
f. incubating each antibody with said pool of tagged chromatin fragments or a random sub-pool thereof,
g. obtaining chromatin fragments binding each antibody, thereby obtaining a sub-pool comprising tagged gDNA fragments from chromatin fragments comprising the chromatin modification recognised by said antibody referred to as a “chromatin modification sub-pool”,
h. optionally amplifying at least a fraction of said tagged gDNA fragments in said chromatin modification sub-pool, thereby obtaining copies of gDNA fragments, wherein said gDNA fragments and said copies thereof collectively are referred to as “gDNA fragments”;
i. Randomly selecting in the range of n times 100 to 100,000 tagged gDNA fragments, from each chromatin modification sub-pool, wherein n is the number of samples provided in step a.; or
pooling tagged gDNA fragments from all chromatin modification sub-pools into a combined pool and randomly selecting in the range of n times m times 100 to 100,000 tagged gDNA fragments from said combined pool, wherein n is the number of samples provided in step a. and m is the number of chromatin modification sub-pools;
j. Sequencing at least part of each of said selected tagged gDNA fragments, and determining the number of unique tagged gDNA fragments comprising each barcode sequence from each chromatin modification sub-pool, wherein
i. at least the first barcode sequence and
ii. the UMI sequence and/or a part of the gDNA sequence is sequenced, and wherein a unique tagged gDNA fragments comprises either a unique UMI and/or a unique gDNA sequence,
k. calculating for each locus of interest the frequency of gDNA fragment comprising each barcode sequence within each chromatin modification sub-pool,
wherein a higher frequency of gDNA fragments comprising a barcode sequence indicates a higher level of said chromatin modification at the locus of interest in the sample tagged with ID-tags comprising said barcode sequence.

2. A method of assessing the local levels of a plurality of chromatin modifications in one or more loci of interest in parallel in a plurality of samples, said method comprising the steps of
a. providing a plurality of test samples comprising chromatin from a cell population comprising a plurality of cells, wherein said samples are physically separated from each other
b. fragmenting chromatin of each sample into chromatin fragments, wherein each chromatin fragment comprises a double-stranded genomic DNA (gDNA) fragment and optionally associated proteins,
c. tagging at least a fraction of the gDNA fragments within each sample with an ID-tag, wherein said ID-tag is an oligonucleotide which comprises a barcode sequence and optionally additional sequences, wherein gDNA fragments within one sample is tagged with a ID-tag comprising the same barcode sequence, and wherein different barcode sequences are used for each sample,
d. combining said tagged chromatin fragments generating a pool of tagged chromatin fragments,
e. providing a plurality of different antibodies, each specifically binding a chromatin modification,
f. incubating each antibody with said pool of tagged chromatin fragments or a random sub-pool thereof,
g. obtaining chromatin fragments binding each antibody, thereby obtaining a sub-pool comprising tagged gDNA fragments from chromatin fragments comprising the chromatin modification recognised by said antibody referred to as a “chromatin modification sub-pool”,
h. amplifying at least a fraction of said tagged gDNA fragments in said chromatin modification sub-pool using at least one primer specific for each locus of interest for said amplification, thereby obtaining copies of gDNA fragments, wherein said gDNA fragments and said copies thereof collectively are referred to as “gDNA fragments”;
i. Randomly selecting in the range of 100 to 100,000 tagged gDNA fragments from each chromatin modification sub-pool, wherein n is the number of samples provided in step a. or
pooling tagged gDNA fragments from all chromatin modification sub-pools into a combined pool and randomly selecting in the range of n times m times 100 to 100,000 tagged gDNA fragments from said combined pool, wherein n is the number of samples provided in step a. and m is the number of chromatin modification sub-pools;
j. Sequencing at least part of each of said selected tagged gDNA fragments, and determining the number of unique tagged gDNA fragments comprising each barcode sequence from each chromatin modification sub-pool,
k. calculating the frequency of gDNA fragment comprising each barcode sequence within each chromatin modification sub-pool,
wherein a higher frequency of gDNA fragments comprising a barcode sequence indicates a higher level of said chromatin modification in the sample tagged with ID-tags comprising said barcode sequence.

3. The method according to any one of the preceding claims, wherein step d. further comprises dividing said pool into random sub-pools, wherein at least one sub-pool is an input sub-pool and the other sub-pools are test sub-pools, and wherein step f. comprises incubating each antibody with a random test sub-pool.

4. The method according to claim 2, wherein said step i. further comprises randomly selecting in the range of n times 100 to 100,000 tagged gDNA fragments from the input sub-pool, and step j. further comprises sequencing the gDNA fragments selected from the input sub-pool, and determining the number of unique gDNA fragments with each barcode sequence from the input sub-pool, and step k. further comprises determining the input normalised read count (INRC) by dividing the frequency of gDNA fragments comprising each barcode sequence within each chromatin modification sub-pool by the frequency of gDNA fragment comprising the same barcode within the input sub-pool, wherein a higher INRC of a barcode sequence indicates a higher level of said chromatin modification in the sample tagged with ID-tags comprising said barcode sequence.

5. The method according to any one of claims 2 to 4, wherein the level of a chromatin modification in sample X compared to the level of chromatin modification in sample Y is determined by the following formula:
$$\frac{\begin{matrix}( Frequency of barcode X in chromatin modification subpool ) / \\ ( Frequency of barcode X in input subpool )\end{matrix}}{\begin{matrix}( Frequency of barcode Y in chromatin modification subpool ) / \\ ( Frequency of barcode Y in input subpool )\end{matrix}}$$
wherein the gDNA fragments of sample X are tagged with an ID-tag comprising barcode X, and the gDNA fragments of sample Y are tagged with an ID-tag comprising barcode Y.

6. The method according to any one of the preceding claims, wherein the ID-tag comprises said barcode sequence and a unique molecular identifier (UMI) sequence, wherein each ID-tag comprises a different UMI sequence.

7. The method according to claim 6, wherein determining the number of unique DNA fragments is done by determining the number of unique UMIs.

8. The methods according to any one of the preceding claims, wherein the method further comprises tagging at least a fraction of the gDNA fragments with a second tag.

9. The methods according to any one of the preceding claims, wherein the method further comprises tagging at least a fraction of the gDNA fragments within each chromatin modification sub-pool with a second tag, wherein said second tag is an oligonucleotide comprising a second barcode sequence, wherein gDNA fragments within one chromatin modification sub-pool is tagged with a second tag comprising the same second barcode sequence, and wherein different second barcode sequences are used for each chromatin modification sub-pool.

10. The method according to claim 9, wherein step j. comprises sequencing at least the barcode sequence of the ID-tag and the second barcode sequence and the UMI sequence and/or the gDNA sequence, and wherein step j. comprises calculating the frequency of unique gDNA fragments comprising the barcode sequence of the ID-tag and each specific second barcode sequence in relation total number of unique gDNA fragments comprising said specific second barcode sequence.

11. The method according to any one of the preceding claims, wherein step a. comprises providing at least 15, such as at least 25, for example at least at least 50, such as at least 75, for example in the range of 15 to 1000, such as in the range of 15 to 500, for example in the range of 25 to 1000, such as in the range of 25 to 500 different test samples comprising chromatin are provided.

12. The method according to any one of the preceding claims, wherein step a. comprises providing at least 75, preferably at least 85, for example in the range of 75 to 1000, such as in the range of 75 to 500, for example providing in the range of 85 to 1000, such as in the range of 85 to 500 different test samples comprising chromatin.

13. The method according to any one of the preceding claims, wherein step e. comprises providing at least 5 different antibodies, such as at least 10 different antibodies, for example at least 15 different antibodies, such as in the range of 5 to 100 different antibodies, for example in the range of 5 to 50 different antibodies, such as in the range of 10 to 100 different antibodies, for example in the range of 10 to 50 different antibodies each specifically binding a different chromatin modification.

14. The method according to any one of the preceding claims, wherein one or more antibodies specifically and selectively binds a posttranslational modification selected from the group consisting of carboxylation, methylation, hydroxymethylation, acetylation, glutamylation, citrullination, phosphorylation and glycosylation of an amino acid.

15. The method according to any of the preceding claims, wherein the method comprises randomly selecting and sequencing of in the range of n times 100 to 100,000, for example at the most n times 50,000, such as at the most n times 20,000, for example in the range of n times 1000 to 50,000, such as in the range of n times 5000 to 20,000 tagged gDNA fragments from each chromatin modification sub-pool per .

16. The method according to any one of the preceding claims, wherein the method comprises randomly selecting and sequencing in the range of n times m times 100 to 100,000, for example at the most n time m times 50,000, such as at the most n time m times 20,000, for example in the range of n time m times 1000 to 50,000, such as in the range of n time m times 5000 to 20,000 tagged gDNA fragments from said combined pool.

17. The method according to any one of the preceding claims, wherein the cell population comprises at least 100 cells, preferably at least 500 cells, even more preferably at least 1000 cells, for example in the range of 10 to 100,000 cells, such as in the range of 100 to 100,000 cells, for example in the range of 1000 to 100,000 cells.

18. A method of determining the influence of test compounds on the level of a plurality of chromatin modifications, said method comprising the steps of
a. Providing one or more test compounds;
b. Cultivating a plurality of cells in the presence of said test compounds or combinations thereof, wherein cells cultivated in the presence of different test compounds or combinations therof are physically separated from each other, and wherein cells cultivated in the presence of a given test compound or combination thereof is a cell population;
c. Performing the method according to any one of claims 1 to 17, wherein each test samples comprises chromatin from different cell populations.

19. The method according to claim 18, wherein the method further comprises performing the method according to any one of claims 1 to 17 with a reference sample comprising cells, which have not been incubated with a test compound.
